(19) United States
(12) Patent Application Publication
(10) Pub. No.: US 2009/0087840 A1
(43) Pub. Date: Apr. 2, 2009

(54) Combined extension and ligation for nucleic acid assembly

(75) Inventors: Brian M. Baynes, Cambridge, MA (US); Brad Chapman, Somerville, MA (US); Lee Kamentsky, Arlington, MA (US)

Correspondence Address: WOLF GREENFIELD & SACKS, P.C., 600 ATLANTIC AVENUE, BOSTON, MA, 02210-2206, US

(73) Assignee: Codon Devices, Inc., Cambridge, MA (US)

(21) Appl. No.: 11/804,996

(22) Filed: May 19, 2007

Related U.S. Application Data
(60) Provisional application No. 60/801,842, filed on May 19, 2006.

(51) Int. Cl. C12Q 1/68; C12M 1/00

(52) U.S. Cl. 435/6; 435/287.2

(57) ABSTRACT

Certain aspects of the present invention provide methods for assembling nucleic acid molecules. Some embodiments involve analyzing nucleic acid sequences and determining appropriate assembly strategies based on the presence or absence of sequence features that are known or predicted to interfere with extension-based and/or ligation-based assembly techniques. Aspects of the invention also provide kits, compositions, devices, and systems for assembling synthetic nucleic acids using polymerase-based techniques, ligase-based techniques, or combinations thereof.

### RELATED APPLICATIONS

This application claims the benefit under 35 U.S.C. §119(e) from U.S. provisional application Ser. No. 60/801,842, filed May 19, 2006, the entire contents of which are herein incorporated by reference.

### FIELD OF THE INVENTION

The invention relates to methods of nucleic acids assembly.

### BACKGROUND

Recombinant and synthetic nucleic acids have many applications in research, industry, agriculture, and medicine. Recombinant and synthetic nucleic acids can be used to express and obtain large amounts of polypeptides, including enzymes, antibodies, growth factors, receptors, and other polypeptides that may be used for a variety of medical, industrial, or agricultural purposes. Recombinant and synthetic nucleic acids also can be used to produce genetically modified organisms including modified bacteria, yeast, mammals, plants, and other organisms. Genetically modified organisms may be used in research (e.g., as animal models of disease, as tools for understanding biological processes, etc.), in industry (e.g., as host organisms for protein expression, as bioreactors for generating industrial products, as tools for environmental remediation, for isolating or modifying natural compounds with industrial applications, etc.), in agriculture (e.g., modified crops with increased yield or increased resistance to disease or environmental stress, etc.), and for other applications. Recombinant and synthetic nucleic acids also may be used as therapeutic compositions (e.g., for modifying gene expression, for gene therapy, etc.) or as diagnostic tools (e.g., as probes for disease conditions, etc.).

Numerous techniques have been developed for modifying existing nucleic acids (e.g., naturally occurring nucleic acids) to generate recombinant nucleic acids. For example, combinations of nucleic acid amplification, mutagenesis, nuclease digestion, ligation, cloning and other techniques may be used to produce many different recombinant nucleic acids. Chemically synthesized polynucleotides are often used as primers or adaptors for nucleic acid amplification, mutagenesis, and cloning.

Techniques also are being developed for de novo nucleic acid assembly whereby nucleic acids are made (e.g., chemically synthesized) and assembled to produce longer target nucleic acids of interest. For example, different multiplex assembly techniques are being developed for assembling oligonucleotides into larger synthetic nucleic acids that can be used in research, industry, agriculture, and/or medicine.

### SUMMARY OF THE INVENTION

Aspects of the invention relate to methods and compositions for assembling nucleic acids using multiplex assembly techniques. Aspects of the invention provide methods for analyzing a nucleic acid sequence of interest to determine whether an assembly strategy should include extension-based assembly techniques, ligation-based assembly techniques, or a combination thereof. In some embodiments, a target nucleic acid sequence is analyzed to determine whether it contains one or more interfering sequence features that could disrupt or interfere with a polymerase-based extension assembly technique. The presence of one or more interfering sequence features may be addressed by i) designing and/or using a polymerase-based assembly that minimizes or avoids the disruptive effects of the interfering features (e.g., the disruptive effect of repeated sequences may be avoided by separating the repeated sequences during assembly, for example by assembling different regions of a target nucleic acid separately wherein each region contains several separate fragments each containing at most a single copy of the sequence that is repeated), and/or ii) designing and/or using a ligation-based technique to assemble regions containing one or more interfering sequence features, or a combination thereof. Aspects of the invention also relate to assembly procedures that involve a combination of polymerase-based and ligation-based assembly steps.

Design and assembly methods of the invention may be automated. Methods of the invention may reduce the cost and increase the speed and accuracy of nucleic acid assembly procedures, particularly automated assembly procedures.

In one aspect, the invention provides a method of synthesizing a target nucleic acid, by analyzing a sequence of a target nucleic acid for the presence of one or more predetermined interfering sequence features that are predicted to disrupt a multiplex oligonucleotide assembly reaction involving polymerase-based extension step, and assembling one or more first fragments of the target nucleic acid using a polymerase-mediated oligonucleotide assembly reaction, wherein no predetermined interfering sequence features are present in any of the one or more first fragments. Further, if one or more predetermined interfering sequence features are present in the target nucleic acid, one or more second fragments of the target nucleic acid are assembled using a ligase-mediated oligonucleotide assembly reaction, wherein the one or more second fragments comprise the one or more predetermined interfering sequence features. The target nucleic acid can be assembled from the one or more first fragments and the one or more second fragments if the target nucleic acid contains one or more predetermined interfering sequence features.

In another aspect the invention provides a method of designing and/or implementing a target nucleic acid assembly procedure by analyzing a sequence of a target nucleic acid for the presence of one or more predetermined interfering sequence features that are predicted to disrupt a multiplex oligonucleotide assembly reaction (e.g. a multiplex assembly reaction including one or more polymerase-based assembly steps), and selecting one or more first fragments of the target nucleic acid for assembly in a polymerase-mediated oligonucleotide assembly reaction, wherein the one or more first fragments are selected so that no predetermined interfering sequence features are present in any of the one or more first fragments. Further, if one or more predetermined interfering sequence features are present in the target nucleic acid, selecting one or more second fragments of the target nucleic acid for assembly in a ligase-mediated oligonucleotide assembly reaction, wherein the one or more second fragments are selected to comprise the one or more predetermined interfering sequence features. The target nucleic acid can be assembled from the one or more first fragments and the one or more second fragments if they are selected.

In some embodiments, the method of designing and/or implementing a target nucleic acid assembly procedure further comprises the act of assembling the one or more first fragments. In some embodiments, the method further comprises the act of assembling the one or more first fragments and the act of assembling the one or more second fragments. In some embodiments, the method further comprises the act of assembling the target nucleic acid.

In some embodiments of these methods, each of the one or more predetermined interfering sequence features is, independently, a region of high GC content, a region of low GC content, a region containing a repeated sequence, a region containing a partially repeated sequence, a region containing a polymerase stall sequence, a region containing a sequence likely to be replicated erroneously by a polymerase, or any other sequence feature predicted to disrupt a multiplex oligonucleotide assembly reaction. In certain embodiments, the region of high GC content contains greater than 70% GC over a length of at least 10 to 20 nucleotides. In certain embodiments, the region of low GC content contains less than 30% GC over a length of at least 10-20 nucleotides. In some embodiments, the repeated sequence is at least 7 bases long. In some embodiments, the repeated sequence is a direct repeat, while in others it is an inverted repeat.

In some embodiments, the act of analyzing the sequence of the target nucleic acid comprises calculating hybridization binding energies between pairs of test sequences selected from different regions of the target nucleic acid. In certain embodiments, each test sequence is a randomly selected sequence of between 20 and 60 consecutive bases of the target nucleic acid. In certain embodiments, each test sequence is selected according to a computer-implemented algorithm. In certain embodiments, an interfering sequence feature is the presence of a pair of test sequences having a predicted hybridization binding energy greater than a threshold level.

In some embodiments, an interfering sequence feature is the presence of a pair of test sequences that are predicted to form a stem-loop structure wherein at most two bases are unpaired at the 3′ end of one of the sequences.

In some embodiments, the act of analyzing the sequence of the target nucleic acid comprises searching for potential stem-loop structures between pairs of test sequences selected from different regions of the target nucleic acid. In some embodiments, each test sequence is a randomly selected sequence of between 20 and 60 consecutive bases of the target nucleic acid. In some embodiments, each test sequence is selected according to a computer-implemented algorithm.

In some embodiments, the act of analyzing the target sequence is computer-implemented. In some embodiments, the act of selecting the one or more first fragments is computer-implemented. In some embodiments, the act of selecting the one or more second fragments is computer-implemented.

In some embodiments, no predetermined interfering sequences are identified and the target nucleic acid is assembled from the one or more first fragments.

In some embodiments, each of the one or more first fragments is may be 200 and 800 nucleotides long. However, shorter or longer fragments may be designed, selected, and/or assembled as the invention is not limited in this respect. In certain embodiments, each of the one or more first fragments is about 400 nucleotides long. In some embodiments, each of one or more first fragments is about 500 nucleotides long.

Similarly, each of the one or more second fragments may be between 200 and 800 nucleotides long. However, shorter or longer second fragments may be designed, selected, and/or assembled. In certain embodiments, each of the one or more second fragments is about 400 nucleotides long. In some embodiments, each of the one or more second fragments may be about 500 nucleotides long.

It should be appreciated that each first and each second fragment may have identical, similar, or different lengths. In some embodiments, the polymerase-mediated oligonucleotide assembly reaction comprises two or more cycles of denaturing, annealing, and extension conditions. In some embodiments, a low processivity polymerase protein is used in the polymerase-mediated oligonucleotide assembly reaction. In some embodiments, each of the one or more first fragments are assembled from between about 5 and 40 different synthetic oligonucleotides. In some embodiments, each of the one or more first fragments are assembled from more than 40 different synthetic oligonucleotides. In some embodiments, the method further comprises the act of amplifying the target nucleic acid. In some embodiments, the method further comprises cloning the target nucleic acid. In some embodiments, the method further comprises transforming a host cell with the target nucleic acid. In certain embodiments, the target nucleic acid is integrated into the genome of the host cell.

In some embodiments, the method further comprises inserting the target nucleic acid into a vector. In certain embodiments, the method further comprises transforming a host cell with the vector, and/or amplifying said vector. In certain embodiments, the vector is amplified in vivo.

In some embodiments, the method further comprises sequencing the target nucleic acid. In some embodiments, the method further comprises expressing a protein from said target nucleic acid. In certain embodiments, the method further comprises isolating from the host cell a polypeptide encoded by the target nucleic acid.

Another aspect of the invention provides a method of producing a target nucleic acid, the method comprising transforming a host cell with a target nucleic acid and propagating the host cell, wherein the target nucleic acid is a product of an assembly process according to one or more of the above-recited methods of synthesizing a target nucleic acid.

Another aspect of the invention provides a host cell comprising a target nucleic acid, wherein the target nucleic acid is a product of an assembly process according to one or more of the methods described herein for synthesizing a target nucleic acid.

Another aspect of the invention provides a vector comprising a target nucleic acid, wherein the target nucleic acid is a product of an assembly process according to one or more of the above-recited methods of synthesizing a target nucleic acid.

Another aspect of the invention provides a method of distributing a nucleic acid molecule, the method comprising providing a host cell as recited herein or a vector as recited above.

Another aspect of the invention provides a method of propagating a target nucleic acid, the method comprising obtaining a target nucleic acid that was assembled in a process comprising one or more of the methods described herein for synthesizing a target nucleic acid, and cloning the target nucleic acid.

Another aspect of the invention provides a method of propagating a target nucleic acid by obtaining a target nucleic acid that was assembled in a process comprising one or more of the methods described herein for synthesizing a target nucleic acid, and transforming a host cell with the target nucleic acid.

Another aspect of the invention provides a method of propagating a target nucleic acid by obtaining a host cell transformed with a target nucleic acid that was produced in a process comprising one or more of the present methods of synthesizing a target nucleic acid, and growing the transformed host cell.

Another aspect of the invention provides a method of propagating a target nucleic acid by obtaining a target nucleic acid that was produced in a process comprising one or more of the present methods of synthesizing a target nucleic acid, and amplifying the target nucleic acid. The target nucleic acid can be amplified in vitro or in vivo (e.g., in a host cell).

Another aspect of the invention provides a method of isolating a polypeptide by obtaining a host cell transformed with a target nucleic acid that was produced in a process comprising one or more of the present methods of synthesizing a target nucleic acid, and isolating, from the host cell, a polypeptide encoded by the target nucleic acid.

Another aspect of the invention provides a method of isolating a polypeptide by obtaining a lysate of a host cell transformed with a target nucleic acid that was produced in a process comprising one or more of the present methods of synthesizing a target nucleic acid, and isolating from the lysate a polypeptide encoded by the target nucleic acid.

Another aspect of the invention provides a method of obtaining a target nucleic acid by sending sequence information for a target nucleic acid to a remote site, and sending delivery information for the target nucleic acid, wherein the target nucleic acid is assembled at the remote site using a process comprising one or more of the present methods of synthesizing a target nucleic acid.

Another aspect of the invention provides a method of obtaining a design for a target nucleic acid assembly process by sending sequence information for a target nucleic acid to a remote site where a method for designing an assembly process for the target nucleic acid is implemented according to one or more of the present methods of designing a target nucleic acid, and receiving from the remote site information relating to the one or more first fragments and the one or more second fragments if the target nucleic acid contains one or more interfering sequence features.

Another aspect of the invention provides a system for assembling a target nucleic acid, the system comprising a means for analyzing a sequence of a target nucleic acid for the presence of one or more predetermined interfering sequence features that are predicted to disrupt a multiplex oligonucleotide assembly reaction, a means for assembling one or more first fragments of the target nucleic acid using a polymerase-mediated oligonucleotide assembly reaction, wherein no predetermined interfering sequence features are present in any of the one or more first fragments, a means for assembling one or more second fragments of the target nucleic acid using a ligase-mediated oligonucleotide assembly reaction if one or more predetermined interfering sequence features are present in the target nucleic acid, wherein the one or more second fragments comprise the one or more predetermined interfering sequence features, and a means for assembling the target nucleic acid from the one or more first and second fragments. In another embodiment, the system is automated using computer-implemented means.

Another aspect of the invention provides a system for designing a target nucleic acid assembly process, the method comprising a means for analyzing a sequence of a target nucleic acid for the presence of one or more predetermined interfering sequence features that are predicted to disrupt a multiplex oligonucleotide assembly reaction, a means for selecting one or more first fragments of the target nucleic acid for assembly in a polymerase-mediated oligonucleotide assembly reaction, wherein no predetermined interfering sequence features are present in any of the one or more first fragments, and a means for selecting one or more second fragments of the target nucleic acid for assembly in a ligase-mediated oligonucleotide assembly reaction if one or more predetermined interfering sequence features are present in the target nucleic acid, wherein the one or more second fragments comprise the one or more predetermined interfering sequence features, wherein the target nucleic acid can be assembled from the one or more first and second fragments.

In some embodiments, these systems are automated using computer-implemented means.

Another aspect of the invention provides a method of automating a multiplex oligonucleotide assembly reaction by assembling one or more first fragments of a target nucleic acid using a polymerase-mediated oligonucleotide assembly reaction, assembling one or more second fragments of a target nucleic acid using a ligase-mediated oligonucleotide assembly reaction, wherein the one or more second fragments comprise an interfering sequence suspected of disrupting a polymerase-mediated oligonucleotide assembly reaction, and wherein no interfering sequences are present in the one or more first fragments, and assembling the one or more first fragments and second fragments. Another aspect of the invention provides a business method comprising marketing the automated multiplex assembly reaction. Another aspect of the invention provides a business method comprising marketing the method of designing a target nucleic acid assembly process.

Accordingly, in some embodiments, the target nucleic acid may be amplified, sequenced or cloned after it is made. In some embodiments, a host cell may be transformed with the assembled target nucleic acid. The target nucleic acid may be integrated into the genome of the host cell. In some embodiments, the target nucleic acid may encode a polypeptide. The polypeptide may be expressed (e.g., under the control of an inducible promoter). The polypeptide may be isolated or purified. A cell transformed with an assembled nucleic acid may be stored, shipped, and/or propagated (e.g., grown in culture).

In another aspect, the invention provides methods of obtaining target nucleic acids by sending sequence information and delivery information to a remote site. The sequence may be analyzed at the remote site. The starting nucleic acids may be designed and/or produced at the remote site. The starting nucleic acids may be assembled in a reaction involving a combination of ligation and extension techniques at the remote site. In some embodiments, the starting nucleic acids, an intermediate product in the assembly reaction, and/or the assembled target nucleic acid may be shipped to the delivery address that was provided.

Other aspects of the invention provide systems for designing starting nucleic acids and/or for assembling the starting nucleic acids to make a target nucleic. Other aspects of the invention relate to methods and devices for automating a multiplex oligonucleotide assembly reaction that involves a combination of ligation and extension assembly techniques. Yet further aspects of the invention relate to business methods of marketing one or more methods, systems, and/or automated procedures that involve a combination of ligation and extension multiplex nucleic acid assembly reactions.

Other features and advantages of the invention will be apparent from the following detailed description, and from the claims. The claims provided below are hereby incorporated into this section by reference.

### BRIEF DESCRIPTION OF THE FIGURES

FIG. 1 illustrates non-limiting aspects of an embodiment of a polymerase-based multiplex oligonucleotide assembly reaction;

FIG. 2 illustrates non-limiting aspects of an embodiment of sequential assembly of a plurality of oligonucleotides in a polymerase-based multiplex assembly reaction;

FIG. 3 illustrates a non-limiting embodiment of a ligase-based multiplex oligonucleotide assembly reaction;

FIG. 4 illustrates several non-limiting embodiments of ligase-based multiplex oligonucleotide assembly reactions on supports; and

FIG. 5 illustrates a non-limiting embodiment of a nucleic acid assembly procedure.

### DETAILED DESCRIPTION OF THE INVENTION

Aspects of the invention relate to methods for designing and implementing nucleic acid assembly strategies that can accommodate sequence features known or predicted to interfere with one or more assembly steps. Aspects of the invention provide methods for analyzing a target nucleic acid sequence and designing a preferred (e.g., optimal) combination of extension-based and/or ligation-based assembly steps for producing a nucleic acid having the target sequence. Aspects of the invention may be useful for optimizing nucleic acid assembly reactions to reduce the number of incorrectly assembled nucleic acids thus facilitating the process of obtaining a target sequence. Accordingly, methods of the invention may increase the probability of obtaining a correctly assembled nucleic acid and thereby reduce the cost and time associated with the production of a nucleic acid having a predetermined sequence. In some embodiments, methods of the invention may be used to assemble nucleic acids that otherwise may be difficult to make (e.g., certain nucleic acids containing repeated sequences, sequences having a significantly high or low GC content, and/or other sequences associated with secondary structures that can interfere with one or more assembly steps).

In one aspect, methods of the invention may be used to determine an optimal assembly strategy for a predetermined nucleic acid. As described herein, nucleic acid fragments may be assembled from smaller nucleic acids using extension-based multiplex assembly reactions, ligation-based multiplex assembly reactions, or a combination thereof.

The terms “multiplex assembly” and “multiplex oligonucleotide assembly reaction” used herein generally refer to assembly reactions involving a plurality of starting nucleic acids (e.g., a plurality of at least partially overlapping nucleic acids) that are assembled to produce a larger final nucleic acid. It should be appreciated that multiplex assembly reactions are often iterative processes involving several cycles of assembly wherein products from a plurality of first assembly reactions are combined in second assembly reactions to produce one or more larger assembled products that may be further combined to produce larger products. This process may be repeated several times depending on the size of the starting nucleic acids and the size of the final target product. At each cycle, nucleic acids may be assembled by ligation, extension, or other suitable technique.

As used herein, “ligation-based multiplex assembly” refers to a mode of multiplex assembly involving ligation of a plurality of nucleic acids.

As used herein, “extension-based multiplex assembly” refers to a mode of multiplex assembly in which at least a portion of a target sequence is generated by extension of a plurality of nucleic acids (e.g., at least partially overlapping oligonucleotides). For example, one mode of extension-based multiplex assembly involves partially overlapping nucleic acid fragments, where one or more single-stranded gaps between such overlapping segments are “filled” by polymerase-dependent extension. In another mode, extension-based assembly involves more traditional polymerase-based extension, such as primer-based PCR. Multiplex assembly may include combinations of different modes of assembly as described in more detail herein.

The present invention is based, at least in part, on the notion that one or more modes of multiplex assembly, or combinations of modes of assembly, may be preferentially utilized to obtain a final target nucleic acid depending, at least in part, on certain sequence features of the final target sequence.

Aspects of the invention may be used to improve the yield of one or more initial or intermediate assembly reactions, thereby improving the efficiency of the overall assembly procedure by avoiding the requirement to repeat or try alternative assembly methods for certain fragments after finding that they are not correctly assembled and/or produced with sufficient yield in a standard multiplex assembly procedure. It should be appreciated that a standard multiplex assembly may be designed to include only polymerase based assembly steps when these are faster and more cost-effective than ligase-based assembly steps. However, according to the invention, certain sequences that are not efficiently assembled by polymerase-based assembly techniques may be assembled efficiently by ligase-based assembly. In some embodiments, the invention provides methods for determining which sequences should be assembled by ligation and which ones can be assembled by extension. By making this initial determination and designing and implementing an assembly procedure in accordance with this determination, the efficiency of the overall assembly procedure is increased by avoiding unnecessary trial and error steps where fragments that are found not to be assembled correctly in an initial extension are assembled in one or more additional steps using ligation or other technique. In the absence of predictable and/or reliable assembly strategies of the invention, trial and error approaches can significantly slow down an extension-based assembly procedure, because low yield and/or accuracy of even one intermediate product delays all subsequent assembly steps that incorporate that intermediate product.

For certain target sequences, it may be preferred that the entire target be assembled by an extension-based multiplex assembly. For others, a ligation-based multiplex method may be a preferred method. Yet in some cases, a combination of ligation and extension may be used for a portion or portions of a target sequence, based on certain features of the sequences that would favor one method or the other. The present invention, therefore, provides strategies for selecting a preferred mode(s) of assembly. A number of factors (criteria) may be considered-in determining the choice of assembly methods. These are discussed in more detail herein.

According to aspects of the invention, certain sequence features may interfere with multiplex extension reactions (e.g., polymerase-based assembly reactions) and/or promote the formation of unwanted assembly products thereby reducing or preventing the assembly of correct nucleic acid products. One or more assembly strategies may be used to avoid or reduce the effects of interfering sequence features.

In some embodiments, if a plurality of interfering sequence features are identified in a target nucleic acid sequence, a useful strategy may involve separating the interfering sequence features during assembly. For example, a target nucleic acid may be assembled in a process involving a plurality of intermediate fragments that are designed to contain only a small number of interfering sequences (e.g., 0, 1, 2, or 3). In some embodiments, each intermediate fragment may contain at most one interfering sequence feature. Accordingly, each intermediate fragment may be assembled efficiently (e.g., in a polymerase-based extension reaction). The intermediate fragments subsequently may be assembled using any appropriate method (e.g., a ligation reaction, a polymerase reaction, or a combination thereof).

In some embodiments, a ligation-based assembly reaction may be used to assemble a target nucleic acid fragment that contains one or more sequence features that are known or predicted to interfere with a polymerase-based assembly reaction. Accordingly, a target nucleic acid may be assembled from a plurality of intermediate fragments (e.g., fragments that are between 200 and 1,000 bases long), wherein each intermediate fragment is assembled using a polymerase-based reaction or a ligase-based reaction depending on whether the intermediate fragment contains an interfering sequence feature. In some embodiments, fragment boundaries are selected in order to isolate interfering sequences in one or a few (e.g., 2, 3, 4, or 5) fragments that are assembled using a ligation based technique. It should be appreciated that the number of fragments required to encompass all of the interfering sequence features may depend on the length of the target nucleic acid being assembled, the distribution of the interfering sequence features across the target nucleic acid, and/or the length of the fragments that are being assembled by ligation. In some embodiments, the fragment sizes and boundaries are chosen in order to assemble fewer than about 50% (e.g., about 45%, 40%, 35%, 30%, 25%, 20%, 15%, 10%, 5%, or fewer) of the fragments by ligation. In some embodiments, one or more fragments assembled by ligation may be amplified in vivo in a host cell (e.g., cloned into a vector and transformed into a host cell) prior to further assembly. In certain embodiments, one or more fragments assembled by ligation may be amplified in vitro (e.g., using an amplification reaction such as a PCR or LCR reaction, etc.) prior to further assembly. For example, each of the fragments assembled by ligation and/or extension may include a “tag” sequence on its 5′ and/or 3′ ends, such that an oligonucleotide corresponding to the 5′ end of a ligation-assembled fragment and/or an oligonucleotide corresponding to the 3′ end of the ligation-assembled fragment can be designed to contain a segment of non-target sequence (e.g., a “tag”), wherein the tag sequences are identical or complementary to specific primers that that can be used as amplification primers (e.g., as PCR primers). Accordingly, the non-target sequences, or tags, can be used to amplify each ligation-assembled fragment and/or polymerase assembled fragment. In some embodiments, two or more intermediate assembled fragments (either assembled by a ligation-based or polymerase-based method) may contain common 5′ non-target sequences (e.g., a 5′ tag) and/or common 3′ non-target sequences (e.g., a 3′ tag). Accordingly, appropriate primer pairs corresponding to the common non-target sequences can be used to amplify such fragments simultaneously (e.g., in parallel or in the same reaction mixture). In some cases, non-target sequences that are common to and are used for amplification of a plurality of oligonucleotides or assembled sequences thereof (e.g., fragments of a target) may be used to amplify two or more different fragments that were assembled in different ligase-based assembly reactions. A primer may include an affinity label such as biotin. The non-target sequences subsequently may be removed from amplified nucleic acids by various methods described elsewhere herein, including, for instance, type IIS restriction enzyme, UDG, or T4 DNA polymerase based techniques. In some embodiments, one or more fragments assembled by ligation may be added to a subsequent assembly reaction (e.g., a subsequent ligation or polymerase based extension reaction) without any intervening amplification. However, it should be appreciated that fragments assembled by ligation may be concentrated and/or purified, regardless of whether they are amplified, prior to further assembly. The remainder of the fragments may be assembled by extension (e.g., in a polymerase-based assembly reaction).

In some embodiments, starting nucleic acids may be designed to “bury” one or more interfering sequence features. For example, the starting nucleic acids may be designed to exclude interfering sequence features from their 5′ and/or 3′ ends. Accordingly, the interfering sequence features may be excluded from complementary overlapping regions between adjacent starting nucleic acids that are designed for use in either an extension-based assembly reaction and/or a ligation-based assembly reaction. This may prevent or reduce interference with sequence-specific hybridization reactions that are important for correct assembly of the starting nucleic acids. In some embodiments, it may be sufficient to exclude an interfering sequence feature from the immediate 3′ and/or 5′ end of a starting nucleic acid. For example, an interfering sequence feature may be located at least one nucleotide in from a 3′ and/or 5′ end, and preferably 2, 3, 4, 5, or more nucleotides (e.g., 5-10, 10-15, 15-20, or more nucleotides) in from a 3′ and/or 5′ end of a starting nucleic acid.

It should be appreciated that a combination of different techniques may be used to assemble a target nucleic acid that contains one or more interfering sequence features. The choice of appropriate strategy may depend, in part, on the length of the target nucleic acid, the number, size, and distribution of interfering sequence features in the target nucleic acid, the size of the starting nucleic acids used for assembly, or any combination thereof.

The presence or absence of interfering sequence features may be considered at several stages during the assembly of a target nucleic acid. It should be appreciated that an assembly reaction may involve several rounds of assembly to generate intermediate products that then may be used as a starting nucleic acid for a subsequent assembly reaction. At each stage a decision may be made as to whether the assembly should be a ligase-based or a polymerase-based assembly. It also should be appreciated that there may be different ways of grouping fragments to be assembled at each stage. Larger target nucleic acids may provide a higher number of acceptable alternative assembly strategies that smaller target nucleic acids.

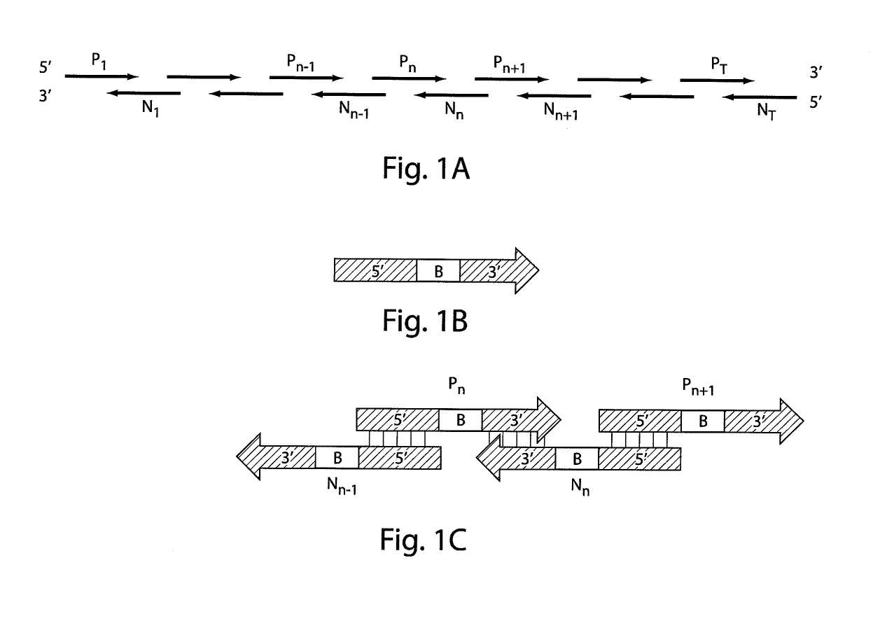

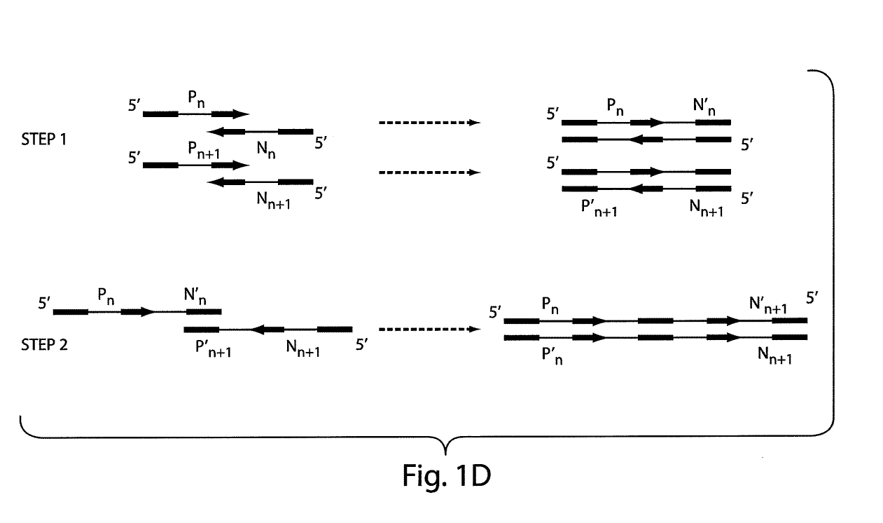

Aspects of the invention may be used in conjunction with in vitro and/or in vivo nucleic acid assembly procedures. Assembly strategies of the invention may involve any suitable extension-based and/or ligation-based assembly reactions. Non-limiting examples of extension-based and ligation-based assembly reactions are described herein and illustrated in FIGS. 1-4.

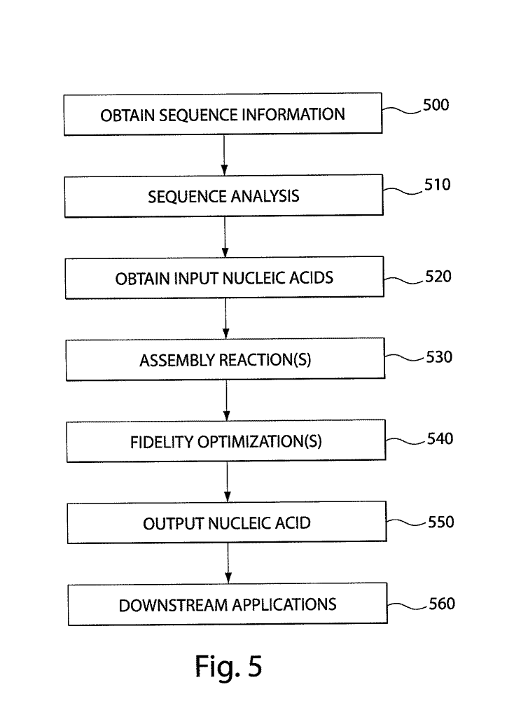

In some embodiments, a sequence analysis and design strategy of the invention may be incorporated in an assembly process outlined in FIG. 5. Ligation steps of the invention may include one or more concerted assembly techniques.

Concerted Assembly

According to aspects of the invention, a plurality of nucleic acid fragments may be assembled in a single concerted procedure wherein the plurality of fragments is mixed together under conditions that promote covalent assembly of the fragments to generate a specific longer nucleic. According to aspects of the invention, a plurality of nucleic acid fragments may be covalently assembled in vivo in a host cell. In some embodiments, a plurality of nucleic acid fragments (e.g., n different nucleic acid fragments) may be mixed together without ligase and transformed into a host cell where they are covalently joined together to produce a longer nucleic acid (e.g., containing the n different nucleic acid fragments covalently liked together). However, a ligase and/or recombinase may be used in some embodiments (e.g., added to a plurality of nucleic acid fragments prior to a host cell transformation). In some embodiments, 5 or more (e.g., 10 or more, 15 or more, 15 to 20, 20 to 25, 25 to 30, 30 to 35, 35 to 40, 40 to 45, 45 to 50, 50 or more, etc.) different nucleic acid fragments may be assembled (e.g., in a concerted in vivo assembly without using ligase). However, it should be appreciated that any number of nucleic acids (e.g., 2, 3, 4, 5, 6, 7, 8, 9, 10, 11, 12, 13, 14, 15, 16, 17, 18, 19, 20, etc.) may be assembled using concerted assembly techniques. Each nucleic acid fragment being assembled may be between about 100 nucleotides long and about 1,000 nucleotides long (e.g., about 200, about 300, about 400, about 500, about 600, about 700, about 800, about 900). However, longer (e.g., about 2,500 or more nucleotides long, about 5,000 or more nucleotides long, about 7,500 or more nucleotides long, about 10,000 or more nucleotides long, etc.) or shorter nucleic acid fragments may be assembled using a concerted assembly technique (e.g., shotgun assembly into a plasmid vector). It should be appreciated that the size of each nucleic acid fragment may be independent of the size of other nucleic acid fragments added to a concerted assembly. However, in some embodiments, each nucleic acid fragment may be approximately the same size (e.g., between about 400 nucleotides long and about 800 nucleotides long). It should be appreciated that the length of a double-stranded DNA fragment may be indicated by the number of base pairs. As used herein, a nucleic acid fragment referred to as “x” nucleotides long corresponds to “x” base pairs in length when used in the context of a double-stranded DNA fragment.

In some embodiments, one or more nucleic acids being assembled in a concerted reaction (e.g., 1-5, 5-10, 10-15, 15-20, etc.) may be codon-optimized and/or non-naturally occurring. In some embodiments, all of the nucleic acids being assembled in a concerted reaction are codon-optimized and/or non-naturally occurring.

In some aspects of the invention, nucleic acid fragments being assembled are designed to have overlapping complementary sequences. In some embodiments, the nucleic acid fragments are double-stranded DNA fragments with 3′ and/or 5′ single-stranded overhangs. These overhangs may be cohesive ends that can anneal to complementary cohesive ends on different DNA fragments. According to aspects of the invention, the presence of complementary sequences (and particularly complementary cohesive ends) on two DNA fragments promotes their covalent assembly in vivo. In some embodiments, a plurality of DNA fragments with different overlapping complementary single-stranded cohesive ends are assembled and their order in the assembled nucleic acid product is determined by the identity of the cohesive ends on each fragment. For example, the nucleic acid fragments may be designed so that a first nucleic acid has a first cohesive end that is complementary to a first cohesive end of the vector and a second cohesive end that is complementary to a first cohesive end of a second nucleic acid. The second cohesive end of the second nucleic acid may be complementary to a first cohesive end of a third nucleic acid. The second cohesive end of the third nucleic acid may be complementary a first cohesive end of a fourth nucleic acid. And so on through to the final nucleic acid that has a first cohesive end that may be complementary to a second cohesive end on the penultimate nucleic acid. The second cohesive end of the final nucleic acid may be complementary to a second cohesive end of the vector. According to aspects of the invention, this technique may be used to generate a vector containing nucleic acid fragments assembled in a predetermined linear order (e.g., first, second, third, forth, . . . , final).

In certain embodiments, the overlapping complementary regions between adjacent nucleic acid fragments are designed (or selected) to be sufficiently different to promote (e.g., thermodynamically favor) assembly of a unique alignment of nucleic acid fragments (e.g., a selected or designed alignment of fragments). It should be appreciated that overlapping regions of different length may be used. In some embodiments, longer cohesive ends may be used when higher numbers of nucleic acid fragments are being assembled. Longer cohesive ends may provide more flexibility to design or select sufficiently distinct sequences to discriminate between correct cohesive end annealing (e.g., involving cohesive ends designed to anneal to each other) and incorrect cohesive end annealing (e.g., between non-complementary cohesive ends).

In some embodiments, two or more pairs of complementary cohesive ends between different nucleic acid fragments may be designed or selected to have identical or similar sequences in order to promote the assembly of products containing a relatively random arrangement (and/or number) of the fragments that have similar or identical cohesive ends. This may be useful to generate libraries of nucleic acid products with different sequence arrangements and/or different copy numbers of certain internal sequence regions.

As illustrated above, each of the two terminal nucleic acid fragments (e.g., the terminal fragment at each end of an assembled product) may be designed to have a cohesive end that is complementary to a cohesive end on a vector (e.g., on a linearized vector). These cohesive ends may be identical cohesive ends that can anneal to identical complementary terminal sequences on a linearized vector. However, in some embodiments the cohesive ends on the terminal fragments are different and the vector contains two different cohesive ends, one at each end of a linearized vector), each complementary to one of the terminal fragment cohesive ends. Accordingly, the vector may be a linearized plasmid that has two cohesive ends, each of which is complementary with one end of the assembled nucleic acid fragments.

In some embodiments, the nucleic acid fragments are mixed with a vector and incubated before transformation into a host cell. It should be appreciated that incubation under conditions that promote specific annealing of the cohesive ends may increase the frequency of assembly (e.g., correct assembly) upon transformation into the host organism. In some embodiments, the different cohesive ends are designed to have similar melting temperatures (e.g., within about 5° C. of each other) so that correct annealing of all of the fragments is promoted under the same conditions. Correct annealing may be promoted at a different temperature depending on the length of the cohesive ends that are used. In some embodiments, cohesive ends of between about 4 and about 30 nucleotides in length (e.g., cohesive ends of about 5, about 10, about 15, about 20, about 25, or about 30 nucleotides in length) may be used. Incubation temperatures may range from about 20° C. to about 50° C. (including, e.g., 37° C.). However, higher or lower temperatures may be used. The length of the incubation may be optimized based on the length of the overhangs, the complexity of the overhangs, and the number of different nucleic acids (and therefore the number of different overhangs) that are mixed together. The incubation time also may depend on the annealing temperature and the presence or absence of other agents in the mixture. For example, a nucleic acid binding protein and/or a recombinase may be added (e.g., RecA, for example a heat stable RecA protein). The resulting complex of nucleic acids may be transformed directly into a host without using a ligase. One or more host functions (e.g., ligation, recombination, any other suitable function, or any combination thereof) then form the covalently linked structure. In some embodiments, a ligase may be added prior to transformation. However, it should be appreciated that the expense of a ligase (including, for example, the expense of storing and dispensing the ligase, e.g., automatically) may be avoided by using a ligase-free concerted assembly method of the invention.

In some embodiments, nucleic acid fragments and a vector are transformed into a host cell without any prior incubation period (other than the time required for mixing the nucleic acids and performing the transformation). In some embodiments, a recombinase (for example RecA, e.g., a thermostable RecA) and/or a nucleic acid binding protein may be mixed with the nucleic acid fragments and the vector, and optionally incubated, prior to transformation into a host cell.

It should be appreciated that a plurality of nucleic acid fragments being assembled all may have complementary 3′ overhangs, complementary 5′ overhangs, or a combination thereof. However, the complementary regions of two nucleic acid fragments that are designed to be adjacent should have the same type of overhang. For example, if nucleic acid “n” has a 5′ overhang at its second end, then nucleic acid “n+1” should have a 5′ overhang at its first end. However, nucleic acid “n+1” may have a 3′ overhang at its second end if nucleic acid “n+2” has a 3′ overhang at its first end. It should be understood that different nucleic acid assembly configurations may be designed and constructed. For example, a concerted assembly may involve multiple copies of certain nucleic acids and single copies of other nucleic acids. In some embodiments, one or more nucleic acid fragments being assembled may have blunt ends. In some embodiments, double-stranded blunt ends may have overlapping identical sequences on nucleic acid fragments that are designed to be adjacent to each other on an assembled nucleic acid product.

Any suitable vector may be used, as the invention is not so limited. For example, a vector may be a plasmid, a bacterial vector, a viral vector, a phage vector, an insect vector, a yeast vector, a mammalian vector, a BAC, a YAC, or any other suitable vector. In some embodiments, a vector may be a vector that replicates in only one type of organism (e.g., bacterial, yeast, insect, mammalian, etc.) or in only one species of organism. Some vectors may have a broad host range. Some vectors may have different functional sequences (e.g., origins or replication, selectable markers, etc.) that are functional in different organisms. These may be used to shuttle the vector (and any nucleic acid fragment(s) that are cloned into the vector) between two different types of organism (e.g., between bacteria and mammals, yeast and mammals, etc.). In some embodiments, the type of vector that is used may be determined by the type of host cell that is chosen.

It should be appreciated that a vector may encode a detectable marker such as a selectable marker (e.g., antibiotic resistance, etc.) so that transformed cells can be selectively grown and the vector can be isolated and any insert can be characterized to determine whether it contains the desired assembled nucleic acid. The insert may be characterized using any suitable technique (e.g., size analysis, restriction fragment analysis, sequencing, etc.). In some embodiments, the presence of a correctly assembly nucleic acid in a vector may be assayed by determining whether a function predicted to be encoded by the correctly assembled nucleic acid is expressed in the host cell.

In some embodiments, host cells that harbor a vector containing a nucleic acid insert may be selected for or enriched by using one or more additional detectable or selectable markers that are only functional if a correct (e.g., designed) terminal nucleic acid fragments is cloned into the vector.

Accordingly, a host cell should have an appropriate phenotype to allow selection for one or more drug resistance markers encoded on a vector (or to allow detection of one or more detectable markers encoded on a vector). However, any suitable host cell type may be used (e.g., prokaryotic, eukaryotic, bacterial, yeast, insect, mammalian, etc.). For example, host cells may be bacterial cells (e.g., Escherichia coli, Bacillus subtilis, Mycobacterium spp., M. tuberculosis, or other suitable bacterial cells), yeast cells (for example, Saccharomyces spp., Picchia spp., Candida spp., or other suitable yeast species, e.g., S. cerevisiae, C. albicans, S. pombe, etc.), Xenopus cells, mouse cells, monkey cells, human cells, insect cells (e.g., SF9 cells and Drosophila cells), worm cells (e.g., Caenorhabditis spp.), plant cells, or other suitable cells, including for example, transgenic or other recombinant cell lines. In addition, a number of heterologous cell lines may be used, such as Chinese Hamster Ovary cells (CHO).

In some embodiments, the type of host cell may be determined by the type of vector that is chosen. A host cell may be modified to have increased activity of one or more ligation and/or recombination functions. In some embodiments, a host cell may be selected on the basis of a high ligation and/or recombination activity. In some embodiments, a host cell may be modified to express (e.g., from the genome or a plasmid expression system) one or more ligase and/or recombinase enzymes.

A host cell may be transformed using any suitable technique (e.g., electroporation, chemical transformation, infection with a viral vector, etc.). Certain host organisms are more readily transformed than others. In some embodiments, all of the nucleic acid fragments and a linearized vector are mixed together and transformed into the host cell in a single step. However, in some embodiments, several transformations may be used to introduce all the fragments and vector into the cell (e.g., several successive transformations using subsets of the fragments). It should be appreciated that the linearized vector is preferably designed to have incompatible ends so that it can only be circularized (and thereby confer resistance to a selectable marker) if the appropriate fragments are cloned into the vector in the designed configuration. This avoids or reduces the occurrence of “empty” vectors after selection.

Single-Stranded Overhangs

Certain aspects of the invention involve double-stranded nucleic acids with single-stranded overhangs. Overhangs may be generated using any suitable technique.

In some embodiments, a double-stranded nucleic acid fragment (e.g., a fragment assembled in a multiplex assembly) may be digested with an appropriate restriction enzyme to generate a terminal single-stranded overhang. In some embodiments, fragments that are designed to be adjacent to each other in an assembled product may be digested with the same enzyme to expose complementary overhangs. In some embodiments, overhangs may be generated using a type IIS restriction enzyme. Type IIS restriction enzymes are enzymes that bind to a double stranded nucleic acid at one site, referred to as the recognition site, and make a single double stranded cut outside of the recognition site. The double stranded cut, referred to as the cleavage site, is generally situated 0-20 bases away from the recognition site. The recognition site is generally about 4-7 bp long. All type IIS restriction enzymes exhibit at least partial asymmetric recognition. Asymmetric recognition means that 5′→3′ recognition sequences are different for each strand of the nucleic acid. The enzyme activity also shows polarity meaning that the cleavage sites are located on only one side of the recognition site. Thus, there is generally only one double stranded cut corresponding to each recognition site. Cleavage generally produces 1-5 nucleotide single-stranded overhangs, with 5′ or 3′ termini, although some enzymes produce blunt ends. Either cut is useful in the context of the invention, although in some instances those producing single-stranded overhangs are produced. To date, ˜80 type IIS enzymes have been identified. Examples include but are not limited to BstF5 I, BtsC I, BsrD I, Bts I, Alw I, Bcc I, BsmA I, Ear I, Mly I (blunt), Ple I, Bmr I, Bsa I, BsmB I, Fau I, Mnl I, Sap I, Bbs I, BciV I, Hph I, Mbo II, BfuA I, BspCN I, BspM I, SfaN I, Hga I, BseR I, Bbv I, Eci I, Fok I, BceA I, BsmF I, BtgZ I, BpuE I, Bsg I, Mme I, BseG I, Bse3D I, BseM I, AcIW I, Alw26 I, Bst6 I, BstMA I, Eam1104 I, Ksp632 I, Pps I, Sch I (blunt), Bfi I, Bso31 I, BspTN I, Eco31 I, Esp3 I, Smu I, Bfu I, Bpi I, BpuA I, BstV2 I, AsuHP I, Acc36 I, Lwe I, Aar I, BseM II, TspDT I, TspGW I, BseX I, BstV1 I, Eco57 I, Eco57M I, Gsu I, and Bcg I. Such enzymes and information regarding their recognition and cleavage sites are available from commercial suppliers such as New England Biolabs, Inc. (Ipswich, Mass., U.S.A.).

In some embodiments, each of a plurality of nucleic acid fragments designed for concerted assembly may have a type IIS restriction site at each end. The type IIS restriction sites may be oriented so that the cleavage sites are internal relative to the recognition sequences. As a result, enzyme digestion exposes an internal sequence (e.g., an overhang within an internal sequence) and removes the recognition sequences from the ends. Accordingly, the same type IIS sites may be used for both ends of all of the nucleic acid fragments being prepared for assembly. However, different type IIS sites also may be used. Two fragments that are designed to be adjacent in an assembled product each may include an identical overlapping terminal sequence and a flanking type IIS site that is appropriately located to expose complementary overhangs within the overlapping sequence upon restriction enzyme digestion. Accordingly, a plurality of nucleic acid fragments may be generated with different complementary overhangs. The restriction site at each end of a nucleic acid fragment may be located such that digestion with the appropriate type IIS enzyme removes the restriction site and exposes a single-stranded region that is complementary to a single-stranded region on a nucleic acid fragment that is designed to be adjacent in the assembled nucleic acid product. In some embodiments, one end of each of the two terminal nucleic acid fragments may be designed to have a single-stranded overhang (e.g., after digestion with an appropriate restriction enzyme) that is complementary to a single-stranded overhang of a linearized vector nucleic acid. Accordingly, the resulting nucleic acid fragments and vector may be transformed directly into a host cell. Alternatively, the nucleic acid fragments and vector may be incubated to promote hybridization and annealing of the complementary sequences prior to transformation in the host cell. It should be appreciated that a vector may be prepared using any one of the techniques described herein or any other suitable technique that produces a single-stranded overhang that would be complementary to an end of one of the terminal nucleic acid fragments.

Enzymatic digestions of DNA with type II or site-specific restriction enzymes typically generate an overhang of four to six nucleotides. These short cohesive ends may be sufficient for ligating two fragments of DNA containing complementary termini. However, when joining multiple DNA fragments together, longer complementary cohesive termini are preferred to facilitate assembly and to ensure specificity. Accordingly, other techniques may be used to expose longer single-stranded overhangs.

In some embodiments, uracil DNA glycosylase (UDG) may be used to hydrolyze a uracil-glycosidic bond in a nucleic acid thereby removing uracil and creating an alkali-sensitive a basic site in the DNA which can be subsequently hydrolyzed by endonuclease, heat or alkali treatment. As a result, a portion of one strand of a double-stranded nucleic acid may be removed thereby exposing the complementary sequence in the form of a single-stranded overhang. This approach requires the deliberate incorporation of one or more uracil bases on one strand of a double-stranded nucleic acid fragment. This may be accomplished, for example, by amplifying a nucleic acid fragment using an amplification primer that contains a 3′ terminal uracil. After treatment with UDG, the region of the primer 5′ to the uracil may be released (e.g., upon dilution, incubation, exposure to mild denaturing conditions, etc.) thereby exposing the complementary sequence as a single-stranded overhang. It should be appreciated that the length of the overhang may be determined by the position of the uracil on the amplifying primer and by the length of the amplifying primer. UDG is commercially available from suppliers such as Roche Applied Science.

In other embodiments, a technique for exposing a single-stranded overhang may involve a polymerase (e.g., T4 DNA polymerase) that has a suitable editing function. For example, T4 DNA polymerase possesses 3′→5′ exonuclease activity. While this activity favors single-stranded regions, it can function, albeit somewhat less efficiently, on blunt ends. Accordingly, in the absence of any exogenous nucleotides, the 3′ ends of a nucleic acid fragment contacted with T4 DNA polymerase will be progressively digested. The 5′ 3′ polymerase activity of T4 may attempt to replace an excised nucleotide. However, by limiting the type of nucleotides available for incorporation, it is possible to avoid incorporation and favor further excision. In some embodiments, progressive excision on a 3′ 5′ strand may be halted at the first occurrence (in the 3′→5′ direction) of one of the four nucleotides by providing that nucleotide in sufficient amounts in the reaction mixture. The presence of the nucleotide in the reaction will result in an equilibrium being reached between the excision of the nucleotide and its re-incorporation by T4. In some embodiments, a single-stranded overhang may be generated at both ends of a nucleic acid fragment (e.g., if each 3′ end does not contain the nucleotide that is added in the T4 polymerase reaction). In some embodiments, the length of the overhang generated at each end is a function of the sequence at each end (e.g., the length of the 3′ sequence that is free of the nucleotide that is added in the T4 polymerase reaction).

In some embodiments, single-stranded overhangs may be generated by incubating a double-stranded nucleic acid with a polymerase that has an editing function (e.g., T4 DNA polymerase) without adding any nucleotides. The length of the overhangs may be a function of the incubation time. Accordingly, suitable incubation conditions (including suitable incubation times, for example) may be determined to obtain suitable average overhangs (e.g., about 10, about 20, about 30, about 40, about 50 nucleotides long, etc.).

Sequence Analysis and Fragment Design and Selection for Concerted Assembly

Aspects of the invention may include analyzing the sequence of a target nucleic acid and designing an assembly strategy based on the identification of regions, within the target nucleic acid sequence, that can be used to generate appropriate cohesive ends (e.g., single-stranded overhangs). These regions may be used to define the ends of fragments that can be assembled (e.g., in a concerted reaction) to generate the target nucleic acid. The fragments can then be provided or made (e.g., in a multiplex assembly reaction). In some embodiments, a target nucleic acid sequence may be analyzed to identify regions that contain at most three different types of nucleotide (i.e., they are missing at least one of G, A, T or C) on one strand of the target nucleic acid. These regions may be used to generate cohesive ends using a polymerase (e.g., T4 DNA polymerase) processing technique described herein. It should be appreciated that the length of a cohesive end is preferably sufficient to provide specificity. For example, cohesive ends may be long enough to have sufficiently different sequences to prevent or reduce mispairing between similar cohesive ends. However, their length is preferably not long enough to stabilize mispairs between similar cohesive sequences. In some embodiments, a length of about 9 to about 15 bases may be used. However, any suitable length may be selected for a region that is to be used to generate a cohesive overhang. The importance of specificity may depend on the number of different fragments that are being assembled simultaneously. Also, the appropriate length required to avoid stabilizing mispaired regions may depend on the conditions used for annealing different cohesive ends.

In some embodiments, a target nucleic acid sequence may be analyzed to identify potential cohesive end regions as follows. One or more regions (e.g., about 9-15 base long regions) free of either G, A, T or C may be identified on one strand of a target nucleic acid. One or more regions (e.g., about 9-15 base regions) free of the complementary nucleotide may be identified on the same strand. For example, regions free of C and regions free of G may be identified on one strand of the target nucleic acid. Alternating regions (e.g., alternating C-free and G-free regions) may be selected to define the ends of nucleic acid fragments to be used for assembly so that both ends of each fragment can be processed to generate cohesive ends. For example, in one embodiment a fragment with a C-free region at one end and a G-free region at the other end of each strand can be processed to generate cohesive overhangs at each end. In this embodiment, the C-free region may be the 3′ region on both strands and the overhang may be generated by adding C to the T4 polymerase reaction. Similar configurations may be used with any one of G, A, T or C.

In some embodiments, alternating regions may be selected if they are separated by distances that define fragments with suitable lengths for the assembly design. In some embodiments, the alternating regions may be separated by about 200 to about 1,500 bases. However, any suitable shorter or longer distance may be selected. For example, the cohesive regions may be separated by about 500 to about 5,000 bases. It should be appreciated that different patterns of alternating regions may be available depending on several factors (e.g., depending on the sequence of the target nucleic acid, the chosen length of the cohesive ends, and the desired fragment length). In some embodiments, if several options are available, the regions may be selected to maximize the sequence differences between different cohesive ends.

Selection of the cohesive regions defines the fragments that will be assembled to generate the target nucleic acid. Accordingly, the fragment size may be between about 200 and about 1,500 base pairs long, between about 500 and about 5,000 bases long, or shorter or longer depending on the target nucleic acid.

The fragments may be generated or obtained using any suitable technique. In some embodiments, each fragment may be assembled (e.g., in a multiplex oligonucleotide assembly reaction) so that it is flanked by double stranded regions that will be used to generate the cohesive single-stranded regions.

A fragment may be amplified in vitro (e.g., by PCR, LCR, etc.). In some embodiments, a fragment may be amplified in vivo. For in vivo amplification, a nucleic acid may be cloned into a vector having suitable flanking restriction sites. The restriction sites may be used to excise a fragment with appropriate end sequences that can be used to generate cohesive ends (e.g., with appropriate single-stranded lengths). In some embodiments, type IIS restriction enzymes may be used to cut out an appropriate fragment. A type IIS restriction site may be provided by the vector into which a nucleic acid is cloned. Alternatively or additionally, a type IIS restriction site may be provided at the end of a nucleic acid that is cloned into a vector (e.g., at the end of a fragment that is assembled in a multiplex oligonucleotide assembly reaction). After amplification in vivo, a type IIS fragment may be isolated and processed as described herein to generate the cohesive ends. It should be appreciated that any type IIS enzyme may be used, provided that its restriction site is placed at a suitable distance from the cohesive region so that the type IIS fragment can be appropriately processed. A fragment may be processed to generate cohesive ends regardless of whether the type IIS digestion generates overhangs or blunt ends. In some embodiments, the overhangs generated by a type IIS enzyme may not be long enough to provide sufficient specificity.

In some embodiments, each fragment is assembled and fidelity optimized to remove error containing nucleic acids (e.g., using one or more post-assembly fidelity optimization techniques described herein) before being processed to generated cohesive ends. In some embodiments, the fidelity optimization may be performed on the synthesized fragments after they are ligated into a first vector used for amplification. However, in some embodiments, the fragments may not be fidelity optimized, or they may be fidelity optimized after treatment to generate cohesive ends.

It should be appreciated that the different nucleic acid fragments that are used to assemble a target nucleic acid may be obtained or synthesized using different techniques. However, in some embodiments they are all produced using the same technique (e.g., assembled in a multiplex oligonucleotide assembly reaction, cloned into a vector, digested with a type IIS enzyme, and processed with T4 DNA polymerase). The resulting fragments may be assembled in a single step concerted reaction and, for example, cloned into a vector that has a selectable marker. The assembly may include an in vitro ligation. However, in some embodiments, the assembly may be an in vivo shotgun assembly wherein the fragments are transformed into a host cell without undergoing an in vitro ligation.

In some embodiments, fragments are amplified in a first vector that has a first selectable marker and are then combined and assembled into a second vector that has a second selectable marker. As a result, selection for the second selectable marker avoids contamination with the first vector. Accordingly, the reactions may be performed in a procedure that does not require removal (e.g., by purification) of the first vector sequence.

FIG. 5 illustrates a method for assembling a nucleic acid in accordance with one embodiment of the invention. Initially, in act 500, sequence information is obtained. The sequence information may be the sequence of a predetermined target nucleic acid that is to be assembled. In some embodiments, the sequence may be received in the form of an order from a customer. The order may be received electronically or on a paper copy. In some embodiments, the sequence may be received as a nucleic acid sequence (e.g., DNA or RNA). In some embodiments, the sequence may be received as a protein sequence. The sequence may be converted into a DNA sequence. For example, if the sequence obtained in act 500 is an RNA sequence, the Us may be replaced with Ts to obtain the corresponding DNA sequence. If the sequence obtained in act 500 is a protein sequence, it may be converted into a DNA sequence using appropriate codons for the amino acids. When choosing codons for each amino acid, consideration may be given to one or more of the following factors: i) using codons that correspond to the codon bias in the organism in which the target nucleic acid may be expressed, ii) avoiding excessively high or low GC or AT contents in the target nucleic acid (for example, above 60% or below 40%; e.g., greater than 65%, 70%, 75%, 80%, 85%, or 90%; or less than 35%, 30%, 25%, 20%, 15%, or 10%), and iii) avoiding sequence features that may interfere with the assembly procedure (e.g., the presence of repeat sequences or stem loop structures). However, these factors may be ignored in some embodiments as the invention is not limited in this respect. Also, aspects of the invention may be used to reduce errors caused by one or more of these factors. Accordingly, a DNA sequence determination (e.g., a sequence determination algorithm or an automated process for determining a target DNA sequence) may omit one or more steps relating to the analysis of the GC or AT content of the target nucleic acid sequence (e.g., the GC or AT content may be ignored in some embodiments) or one or more steps relating to the analysis of certain sequence features (e.g., sequence repeats, inverted repeats, etc.) that could interfere with an assembly reaction performed under standard conditions but may not interfere with an assembly reaction including one or more concerted assembly steps.

In act 510, the sequence information may be analyzed to determine an assembly strategy. This may involve determining whether the target nucleic acid will be assembled as a single fragment or if several intermediate fragments will be assembled separately and then combined in one or more additional rounds of assembly to generate the target nucleic acid.

A sequence analysis may involve scanning for the presence of one or more interfering sequence features that are known or predicted to interfere with a polymerase-based assembly. According to the invention, an interfering sequence feature reduces the yield of correctly assembled nucleic acid fragments to below a threshold level of correctly assembled product that is expected given the amount of starting nucleic acid used in the assembly reaction. Accordingly, a reference threshold level may be determined using the amount of assembled product obtained for a control fragment (e.g., a fragment known to be efficiently assembled or assembled with sufficient accuracy and/or yield for use in subsequent assembly steps) using a polymerase-based assembly reaction as a reference.

It should be appreciated that any efficiently assembled fragment may be used as a control fragment (e.g., a fragment assembled in the examples provided herein).

An interfering sequence feature that reduces yield of correctly assembled fragments may reduce overall yield of an assembly step or may result in high levels of incorrectly assembled fragments without significantly reducing overall amounts of nucleic acid that is assembled in the step.

According to the invention, certain sequence features reduce the yield of polymerase-mediated assembly products to below a reference threshold (e.g., to below about 75%, below about 50%, below about 25%, below about 10%, below about 5%, below about 1%, or lower fractions of the threshold).

In some embodiments, an interfering sequence feature may be the presence of high GC content (e.g., a GC content of greater than 50%, greater than 60%, greater than 70%, greater than 80% or greater than 90%) over a length of 10 or more bases, for example, 10-20, 20-50, 50-100 or more than 100 bases. In one embodiment, a region of a target nucleic acid with a high GC content should be assembled in a ligase-based reaction.

In some embodiments, an interfering sequence structure may be a sequence that has a low GC content (e.g., less than 30% GC, less than 20% GC, less than 10% GC, etc.) over a length of at least 10 bases (e.g., 10-20, 20-50, 50-100, or more than 100 bases). In one embodiment, a region of a target nucleic acid having a low GC content should be assembled in a ligase-based reaction.

In some embodiments, the sequence of a target nucleic acid may be analyzed to identify regions that are predicted to hybridize to each other with a binding energy that is predicted to interfere with polymerase assembly (e.g., by forming one or more secondary structures that interfere with polymerase mediated extension). The threshold binding energy may be determined based on binding energies associated with sequences known to interfere with polymerase assembly (e.g., that reduce yield to below 50%, 50-10%, below 10%, or below 5% of a threshold yield). An analysis may involve determining whether such interfering hybridizing sequences (e.g., an interfering pair of sequences predicted to hybridize with sufficient binding energy to interfere with extension) are present within any stretch of a predetermined length of a target nucleic acid (e.g., any stretch of about 25-50, about 50-75, about 75-100, about 100-150, about 150-200, or longer or shorter stretches of consecutive bases within a target sequence).

In some embodiments, interfering groups (e.g., pairs, etc.) of hybridizing sequences may be identified as sequences capable of forming stem-loop structures. In certain embodiments, sequences capable of forming stem-loop structures having less than 3, less than 2, or less than 1 unpaired bases within the 3′ end (e.g., within the 5, 6, 7, 8, 9, or 10 bases at the 3′ end of one of the sequences capable of forming a stem-loop structure).

If present, interfering groups (e.g., pairs) of hybridizing sequences may be separated by designing an assembly technique such that members of a group of interfering sequences are initially assembled on separate fragments. In some embodiments, one or more fragments of a target nucleic acid may be assembled by ligation if they are identified as containing interfering groups of hybridizing sequences.

In some embodiments, an interfering sequence structure may be the presence of two or more identical or homologous sequences that are repeated (e.g., direct or inverted repeats) within a fragment (e.g., a fragment between about 200 and about 1,000 bases long, or a longer or shorter fragment) that is to be assembled in a single reaction. The length of interfering repeats may be as short as 6 or 7 bases. However, repeats that are 8, 9, 10, 11, 12 or more bases long or longer (e.g., 15-20, 20-50, etc.) may be more interfering. In some embodiments, a fragment containing interfering repeats is assembled using a ligase-based assembly. Alternatively, the design of the fragments being assembled in a single step may be changed to avoid the presence of two identical or homologous sequences within the same assembly fragment. It should be appreciated that computer-implemented techniques may be used to analyze target sequences and determine whether one or more interfering sequences are present. Programs for predicting certain structures, identifying repeats, and/or calculating binding energies of hybridization are known and can be adapted for applications of the invention according to the parameters described herein. In some embodiments, information relating to structural features that are predicted to be interfering can be provided or used (e.g., in the form of a remotely-accessible database or a databases on computer-readable medium, etc.) to design and/or implement an appropriate assembly strategy.

Accordingly, a design stage may involve determining whether one or more interfering sequence features are present in a target nucleic acid (or fragment thereof), determining whether assembly problems due to any or all of the interfering sequence features can be avoided by choosing initial and/or intermediate assembly fragments so that certain sequences predicted to interfere with each other are assembled on separate fragments (at least for the first one, two, three or more initial polymerase-based assembly cycles). Alternatively, or in addition, interfering sequences may be assembled on a fragment that is assembled by ligation in one of the initial cycles of assembly. According to aspects of the invention, ligation reactions are less susceptible to sequence features that can interfere with polymerase-based assembly reactions. Accordingly, the design stage may involve determining the identity (e.g., the length and boundaries) of the initial and intermediate fragments that are going to be assembled, and/or determining which fragments are to be assembled by ligation and which fragments are to be assembled by extension. It should be appreciated that interfering sequences, if present, will be assembled onto a single fragment at some stage during the assembly of a final target nucleic acid. However, sequence features described herein that may interfere with polymerase-based assembly of initial or intermediate fragments may have less of an effect on polymerase-based assembly of larger fragments. Accordingly, sequences that are separated and/or assembled by ligation during the initial assembly steps during the assembly of pools of starting fragments of between about 20 nucleotides to about 250 nucleotides long (e.g., oligonucleotides of about 50, about 100, about 200, etc., nucleotides long) may subsequently be assembly by polymerase-based extension techniques when they are embedded in longer fragments (e.g., longer intermediate fragments). Accordingly, in some embodiments, assembled intermediate fragments containing interfering sequence features may be further assembled using polymerase based techniques if the intermediate fragments are larger than about 400, than about 500, than about 1,000, or more nucleotides long. In some embodiments, smaller intermediate fragments containing interfering sequences may be assembled by polymerase-based techniques if the interfering sequences are buried within the nucleic acid fragments being assembled as described herein.

Once the overall assembly strategy has been determined, input nucleic acids (e.g., oligonucleotides) for assembling the one or more nucleic acid fragments may be designed. The sizes and numbers of the input nucleic acids may be based in part on the type of assembly reaction (e.g., the type of polymerase-based assembly, ligase-based assembly, chemical assembly, or combination thereof) that is being used for each fragment. The input nucleic acids also may be designed to avoid 5′ and/or 3′ regions that may cross-react incorrectly and be assembled to produce undesired nucleic acid fragments. Other structural and/or sequence factors also may be considered when designing the input nucleic acids. In certain embodiments, some of the input nucleic acids may be designed to incorporate one or more specific sequences (e.g., primer binding sequences, restriction enzyme sites, etc.) at one or both ends of the assembled nucleic acid fragment.

In act 520, the input nucleic acids are obtained. These may be synthetic oligonucleotides that are synthesized on-site or obtained from a different site (e.g., from a commercial supplier). In some embodiments, one or more input nucleic acids may be amplification products (e.g., PCR products), restriction fragments, or other suitable nucleic acid molecules. Synthetic oligonucleotides may be synthesized using any appropriate technique as described in more detail herein. It should be appreciated that synthetic oligonucleotides often have sequence errors. Accordingly, oligonucleotide preparations may be selected or screened to remove error-containing molecules as described in more detail herein.

In act 530, an assembly reaction may be performed for each nucleic acid fragment. For each fragment, the input nucleic acids may be assembled using any appropriate assembly technique (e.g., a polymerase-based assembly, a ligase-based assembly, a chemical assembly, or any other multiplex nucleic acid assembly technique, or any combination thereof). An assembly reaction may result in the assembly of a number of different nucleic acid products in addition to the predetermined nucleic acid fragment. Accordingly, in some embodiments, an assembly reaction may be processed to remove incorrectly assembled nucleic acids (e.g., by size fractionation) and/or to enrich correctly assembled nucleic acids (e.g., by amplification, optionally followed by size fractionation). In some embodiments, correctly assembled nucleic acids may be amplified (e.g., in a PCR reaction) using primers that bind to the ends of the predetermined nucleic acid fragment. In some embodiments the assembled nucleic acids may be ligated into a vector and amplified in a host cell. It should be appreciated that act 530 may be repeated one or more times. For example, in a first round of assembly a first plurality of input nucleic acids (e.g., oligonucleotides) may be assembled to generate a first nucleic acid fragment. In a second round of assembly, the first nucleic acid fragment may be combined with one or more additional nucleic acid fragments and used as starting material for the assembly of a larger nucleic acid fragment. In a third round of assembly, this larger fragment may be combined with yet further nucleic acids and used as starting material for the assembly of yet a larger nucleic acid. This procedure may be repeated as many times as needed for the synthesis of a target nucleic acid. Accordingly, progressively larger nucleic acids may be assembled. At each stage, nucleic acids of different sizes may be combined. At each stage, the nucleic acids being combined may have been previously assembled in a multiplex assembly reaction. However, at each stage, one or more nucleic acids being combined may have been obtained from different sources (e.g., PCR amplification of genomic DNA or cDNA, restriction digestion of a plasmid or genomic DNA, or any other suitable source). It should be appreciated that nucleic acids generated in each cycle of assembly may contain sequence errors if they incorporated one or more input nucleic acids with sequence error(s). Accordingly, a fidelity optimization procedure may be performed after a cycle of assembly in order to remove or correct sequence errors. It should be appreciated that fidelity optimization may be performed after each assembly reaction when several successive cycles of assembly are performed. However, in certain embodiments fidelity optimization may be performed only after a subset (e.g., 2 or more) of successive assembly reactions are complete. In some embodiments, no fidelity optimization is performed.

Accordingly, act 540 is an optional fidelity optimization procedure. Act 540 may be used in some embodiments to remove nucleic acid fragments that seem to be correctly assembled (e.g., based on their size or restriction enzyme digestion pattern) but that may have incorporated input nucleic acids containing sequence errors as described herein. For example, since synthetic oligonucleotides may contain incorrect sequences due to errors introduced during oligonucleotide synthesis, it may be useful to remove nucleic acid fragments that have incorporated one or more error-containing oligonucleotides during assembly. In some embodiments, one or more assembled nucleic acid fragments may be sequenced to determine whether they contain the predetermined sequence or not. This procedure allows fragments with the correct sequence to be identified. However, in some embodiments, other techniques may be used to remove error containing nucleic acid fragments. It should be appreciated that error containing-nucleic acids may be double-stranded homoduplexes having the error on both strands (i.e., incorrect complementary nucleotide(s), deletion(s), or addition(s) on both strands), because the assembly procedure may involve one or more rounds of polymerase extension (e.g., during assembly or after assembly to amplify the assembled product) during which an input nucleic acid containing an error may serve as a template thereby producing a complementary strand with the complementary error. In certain embodiments, a preparation of double-stranded nucleic acid fragments may be suspected to contain a mixture of nucleic acids that have the correct sequence and nucleic acids that incorporated one or more sequence errors during assembly. In some embodiments, sequence errors may be removed using a technique that involves denaturing and reannealing the double-stranded nucleic acids. In some embodiments, single strands of nucleic acids that contain complementary errors may be unlikely to reanneal together if nucleic acids containing each individual error are present in the nucleic acid preparation at a lower frequency than nucleic acids having the correct sequence at the same position. Rather, error containing single strands may reanneal with a complementary strand that contains no errors or that contains one or more different errors. As a result, error-containing strands may end up in the form of heteroduplex molecules in the reannealed reaction product. Nucleic acid strands that are error-free may reanneal with error-containing strands or with other error-free strands. Reannealed error-free strands form homoduplexes in the reannealed sample. Accordingly, by removing heteroduplex molecules from the reannealed preparation of nucleic acid fragments, the amount or frequency of error containing nucleic acids may be reduced. Any suitable method for removing heteroduplex molecules may be used, including chromatography, electrophoresis, selective binding of heteroduplex molecules, etc. In some embodiments, mismatch binding proteins that selectively (e.g., specifically) bind to heteroduplex nucleic acid molecules may be used. One example includes using MutS, a MutS homolog, or a combination thereof to bind to heteroduplex molecules. In E. coli, the MutS protein, which appears to function as a homodimer, serves as a mismatch recognition factor. In eukaryotes, at least three MutS Homolog (MSH) proteins have been identified; namely, MSH2, MSH3, and MSH6, and they form heterodimers. For example in the yeast, Saccharomyces cerevisiae, the MSH2-MSH6 complex (also known as MutSα) recognizes base mismatches and single nucleotide insertion/deletion loops, while the MSH2-MSH3 complex (also known as MutSβ) recognizes insertions/deletions of up to 12-16 nucleotides, although they exert substantially redundant functions. A mismatch binding protein may be obtained from recombinant or natural sources. A mismatch binding protein may be heat-stable. In some embodiments, a thermostable mismatch binding protein from a thermophilic organism may be used. Examples of thermostable DNA mismatch binding proteins include, but are not limited to: Tth MutS (from Thermus thermophilus); Taq MutS (from Thermus aquaticus); Apy MutS (from Aquifex pyrophilus); Tma MutS (from Thermotoga maritima); any other suitable MutS; or any combination of two or more thereof.

According to aspects of the invention, protein-bound heteroduplex molecules (e.g., heteroduplex molecules bound to one or more MutS proteins) may be removed from a sample using any suitable technique (binding to a column, a filter, a nitrocellulose filter, etc., or any combination thereof). It should be appreciated that this procedure may not be 100% efficient. Some errors may remain for at least one of the following reasons. Depending on the reaction conditions, not all of the double-stranded error-containing nucleic acids may be denatured. In addition, some of the denatured error-containing strands may reanneal with complementary error-containing strands to form an error containing homoduplex. Also, the MutS/heteroduplex interaction and the MutS/heteroduplex removal procedures may not be 100% efficient. Accordingly, in some embodiments the fidelity optimization act 540 may be repeated one or more times after each assembly reaction. For example, 2, 3, 4, 5, 6, 7, 8, 9, 10 or more cycles of fidelity optimization may be performed after each assembly reaction. In some embodiments, the nucleic acid is amplified after each fidelity optimization procedure. It should be appreciated that each cycle of fidelity optimization will remove additional error-containing nucleic acid molecules. However, the proportion of correct sequences is expected to reach a saturation level after a few cycles of this procedure.

In some embodiments, the size of an assembled nucleic acid that is fidelity optimized (e.g., using MutS or a MutS homolog) may be determined by the expected number of sequence errors that are suspected to be incorporated into the nucleic acid during assembly. For example, an assembled nucleic acid product should include error free nucleic acids prior to fidelity optimization in order to be able to enrich for the error free nucleic acids. Accordingly, error screening (e.g., using MutS or a MutS homolog) should be performed on shorter nucleic acid fragments when input nucleic acids have higher error rates. In some embodiments, one or more nucleic acid fragments of between about 200 and about 800 nucleotides (e.g., about 200, about 300, about 400, about 500, about 600, about 700 or about 800 nucleotides in length) are assembled prior to fidelity optimization. After assembly, the one or more fragments may be exposed to one or more rounds of fidelity optimization as described herein. In some embodiments, several assembled fragments may be ligated together (e.g., to produce a larger nucleic acid fragment of between about 1,000 and about 5,000 bases in length, or larger), and optionally cloned into a vector, prior to fidelity optimization as described herein.

At act 550, an output nucleic acid is obtained. As discussed herein, several rounds of act 530 and/or 540 may be performed to obtain the output nucleic acid, depending on the assembly strategy that is implemented. The output nucleic acid may be amplified, cloned, stored, etc., for subsequent uses at act 560. In some embodiments, an output nucleic acid may be cloned with one or more other nucleic acids (e.g., other output nucleic acids) for subsequent applications. Subsequent applications may include one or more research, diagnostic, medical, clinical, industrial, therapeutic, environmental, agricultural, or other uses.

Aspects of the invention may include automating one or more acts described herein. For example, sequence analysis, the identification of interfering sequence features, assembly strategy selection (including fragment design and selection, the choice of a particular combination of extension-based and ligation-based assembly reactions, etc.), fragment production, single-stranded overhang production, and/or concerted assembly may be automated in order to generate the desired product automatically. Acts of the invention may be automated using, for example, a computer system.

Aspects of the invention may be used in conjunction with any suitable multiplex nucleic acid assembly procedure. For example, concerted assembly steps may be used in connection with or more of the multiplex nucleic acid assembly procedures described below.

### Multiplex Nucleic Acid Assembly

In aspects of the invention, multiplex nucleic acid assembly relates to the assembly of a plurality of nucleic acids to generate a longer nucleic acid product. In one aspect, multiplex oligonucleotide assembly relates to the assembly of a plurality of oligonucleotides to generate a longer nucleic acid molecule. However, it should be appreciated that other nucleic acids (e.g., single or double-stranded nucleic acid degradation products, restriction fragments, amplification products, naturally occurring small nucleic acids, other polynucleotides, etc.) may be assembled or included in a multiplex assembly reaction (e.g., along with one or more oligonucleotides) in order to generate an assembled nucleic acid molecule that is longer than any of the single starting nucleic acids (e.g., oligonucleotides) that were added to the assembly reaction. In certain embodiments, one or more nucleic acid fragments that each were assembled in separate multiplex assembly reactions (e.g., separate multiplex oligonucleotide assembly reactions) may be combined and assembled to form a further nucleic acid that is longer than any of the input nucleic acid fragments. In certain embodiments, one or more nucleic acid fragments that each were assembled in separate multiplex assembly reactions (e.g., separate multiplex oligonucleotide assembly reactions) may be combined with one or more additional nucleic acids (e.g., single or double-stranded nucleic acid degradation products, restriction fragments, amplification products, naturally occurring small nucleic acids, other polynucleotides, etc.) and assembled to form a further nucleic acid that is longer than any of the input nucleic acids.

In aspects of the invention, one or more multiplex assembly reactions may be used to generate target nucleic acids having predetermined sequences. In one aspect, a target nucleic acid may have a sequence of a naturally occurring gene and/or other naturally occurring nucleic acid (e.g., a naturally occurring coding sequence, regulatory sequence, non-coding sequence, chromosomal structural sequence such as a telomere or centromere sequence, etc., any fragment thereof or any combination of two or more thereof). In another aspect, a target nucleic acid may have a sequence that is not naturally-occurring. In one embodiment, a target nucleic acid may be designed to have a sequence that differs from a natural sequence at one or more positions. In other embodiments, a target nucleic acid may be designed to have an entirely novel sequence. However, it should be appreciated that target nucleic acids may include one or more naturally occurring sequences, non-naturally occurring sequences, or combinations thereof.

In one aspect of the invention, multiplex assembly may be used to generate libraries of nucleic acids having different sequences. In some embodiments, a library may contain nucleic acids having random sequences. In certain embodiments, a predetermined target nucleic acid may be designed and assembled to include one or more random sequences at one or more predetermined positions.

In certain embodiments, a target nucleic acid may include a functional sequence (e.g., a protein binding sequence, a regulatory sequence, a sequence encoding a functional protein, etc., or any combination thereof). However, some embodiments of a target nucleic acid may lack a specific functional sequence (e.g., a target nucleic acid may include only non-functional fragments or variants of a protein binding sequence, regulatory sequence, or protein encoding sequence, or any other non-functional naturally-occurring or synthetic sequence, or any non-functional combination thereof). Certain target nucleic acids may include both functional and non-functional sequences. These and other aspects of target nucleic acids and their uses are described in more detail herein.

A target nucleic acid may be assembled in a single multiplex assembly reaction (e.g., a single oligonucleotide assembly reaction). However, a target nucleic acid also may be assembled from a plurality of nucleic acid fragments, each of which may have been generated in a separate multiplex oligonucleotide assembly reaction. It should be appreciated that one or more nucleic acid fragments generated via multiplex oligonucleotide assembly also may be combined with one or more nucleic acid molecules obtained from another source (e.g., a restriction fragment, a nucleic acid amplification product, etc.) to form a target nucleic acid. In some embodiments, a target nucleic acid that is assembled in a first reaction may be used as an input nucleic acid fragment for a subsequent assembly reaction to produce a larger target nucleic acid.

Accordingly, different strategies may be used to produce a target nucleic acid having a predetermined sequence. For example, different starting nucleic acids (e.g., different sets of predetermined nucleic acids) may be assembled to produce the same predetermined target nucleic acid sequence. Also, predetermined nucleic acid fragments may be assembled using one or more different in vitro and/or in vivo techniques. For example, nucleic acids (e.g., overlapping nucleic acid fragments) may be assembled in an in vitro reaction using an enzyme (e.g., a ligase and/or a polymerase) or a chemical reaction (e.g., a chemical ligation) or in vivo (e.g., assembled in a host cell after transfection into the host cell), or a combination thereof. Similarly, each nucleic acid fragment that is used to make a target nucleic acid may be assembled from different sets of oligonucleotides. Also, a nucleic acid fragment may be assembled using an in vitro or an in vivo technique (e.g., an in vitro or in vivo polymerase, recombinase, and/or ligase based assembly process). In addition, different in vitro assembly reactions may be used to produce a nucleic acid fragment. For example, an in vitro oligonucleotide assembly reaction may involve one or more polymerases, ligases, other suitable enzymes, chemical reactions, or any combination thereof.

### Multiplex Oligonucleotide Assembly

A predetermined nucleic acid fragment may be assembled from a plurality of different starting nucleic acids (e.g., oligonucleotides) in a multiplex assembly reaction (e.g., a multiplex enzyme-mediated reaction, a multiplex chemical assembly reaction, or a combination thereof). Certain aspects of multiplex nucleic acid assembly reactions are illustrated by the following description of certain embodiments of multiplex oligonucleotide assembly reactions. It should be appreciated that the description of the assembly reactions in the context of oligonucleotides is not intended to be limiting. The assembly reactions described herein may be performed using starting nucleic acids obtained from one or more different sources (e.g., synthetic or natural polynucleotides, nucleic acid amplification products, nucleic acid degradation products, oligonucleotides, etc.). The starting nucleic acids may be referred to as assembly nucleic acids (e.g., assembly oligonucleotides). As used herein, an assembly nucleic acid has a sequence that is designed to be incorporated into the nucleic acid product generated during the assembly process. However, it should be appreciated that the description of the assembly reactions in the context of single-stranded nucleic acids is not intended to be limiting. In some embodiments, one or more of the starting nucleic acids illustrated in the figures and described herein may be provided as double stranded nucleic acids. Accordingly, it should be appreciated that where the figures and description illustrate the assembly of single-stranded nucleic acids, the presence of one or more complementary nucleic acids is contemplated. Accordingly, one or more double-stranded complementary nucleic acids may be included in a reaction that is described herein in the context of a single-stranded assembly nucleic acid. However, in some embodiments the presence of one or more complementary nucleic acids may interfere with an assembly reaction by competing for hybridization with one of the input assembly nucleic acids. Accordingly, in some embodiments an assembly reaction may involve only single-stranded assembly nucleic acids (i.e., the assembly nucleic acids may be provided in a single-stranded form without their complementary strand) as described or illustrated herein. However, in certain embodiments the presence of one or more complementary nucleic acids may have no or little effect on the assembly reaction. In some embodiments, complementary nucleic acid(s) may be incorporated during one or more steps of an assembly. In yet further embodiments, assembly nucleic acids and their complementary strands may be assembled under the same assembly conditions via parallel assembly reactions in the same reaction mixture. In certain embodiments, a nucleic acid product resulting from the assembly of a plurality of starting nucleic acids may be identical to the nucleic acid product that results from the assembly of nucleic acids that are complementary to the starting nucleic acids (e.g., in some embodiments where the assembly steps result in the production of a double-stranded nucleic acid product). As used herein, an oligonucleotide may be a nucleic acid molecule comprising at least two covalently bonded nucleotide residues. In some embodiments, an oligonucleotide may be between 10 and 1,000 nucleotides long. For example, an oligonucleotide may be between 10 and 500 nucleotides long, or between 500 and 1,000 nucleotides long. In some embodiments, an oligonucleotide may be between about 20 and about 100 nucleotides long (e.g., from about 30 to 90, 40 to 85, 50 to 80, 60 to 75, or about 65 or about 70 nucleotides long), between about 100 and about 200, between about 200 and about 300 nucleotides, between about 300 and about 400, or between about 400 and about 500 nucleotides long. However, shorter or longer oligonucleotides may be used. An oligonucleotide may be a single-stranded nucleic acid. However, in some embodiments a double-stranded oligonucleotide may be used as described herein. In certain embodiments, an oligonucleotide may be chemically synthesized as described in more detail below.

In some embodiments, an input nucleic acid (e.g., oligonucleotide) may be amplified before use. The resulting product may be double-stranded. In some embodiments, one of the strands of a double-stranded nucleic acid may be removed before use so that only a predetermined single strand is added to an assembly reaction.

In certain embodiments, each oligonucleotide may be designed to have a sequence that is identical to a different portion of the sequence of a predetermined target nucleic acid that is to be assembled. Accordingly, in some embodiments each oligonucleotide may have a sequence that is identical to a portion of one of the two strands of a double-stranded target nucleic acid. For clarity, the two complementary strands of a double stranded nucleic acid are referred to herein as the positive (P) and negative (N) strands. This designation is not intended to imply that the strands are sense and anti-sense strands of a coding sequence. They refer only to the two complementary strands of a nucleic acid (e.g., a target nucleic acid, an intermediate nucleic acid fragment, etc.) regardless of the sequence or function of the nucleic acid. Accordingly, in some embodiments a P strand may be a sense strand of a coding sequence, whereas in other embodiments a P strand may be an anti-sense strand of a coding sequence. According to the invention, a target nucleic acid may be either the P strand, the N strand, or a double-stranded nucleic acid comprising both the P and N strands.

It should be appreciated that different oligonucleotides may be designed to have different lengths. In some embodiments, one or more different oligonucleotides may have overlapping sequence regions (e.g., overlapping 5′ regions or overlapping 3′ regions). Overlapping sequence regions may be identical (i.e., corresponding to the same strand of the nucleic acid fragment) or complementary (i.e., corresponding to complementary strands of the nucleic acid fragment). The plurality of oligonucleotides may include one or more oligonucleotide pairs with overlapping identical sequence regions, one or more oligonucleotide pairs with overlapping complementary sequence regions, or a combination thereof. Overlapping sequences may be of any suitable length. For example, overlapping sequences may encompass the entire length of one or more nucleic acids used in an assembly reaction. Overlapping sequences may be between about 5 and about 500 nucleotides long (e.g., between about 10 and 100, between about 10 and 75, between about 10 and 50, about 20, about 25, about 30, about 35, about 40, about 45, about 50, etc.) However, shorter, longer or intermediate overlapping lengths may be used. It should be appreciated that overlaps between different input nucleic acids used in an assembly reaction may have different lengths.

In a multiplex oligonucleotide assembly reaction designed to generate a predetermined nucleic acid fragment, the combined sequences of the different oligonucleotides in the reaction may span the sequence of the entire nucleic acid fragment on either the positive strand, the negative strand, both strands, or a combination of portions of the positive strand and portions of the negative strand. The plurality of different oligonucleotides may provide either positive sequences, negative sequences, or a combination of both positive and negative sequences corresponding to the entire sequence of the nucleic acid fragment to be assembled. In some embodiments, the plurality of oligonucleotides may include one or more oligonucleotides having sequences identical to one or more portions of the positive sequence, and one or more oligonucleotides having sequences that are identical to one or more portions of the negative sequence of the nucleic acid fragment. One or more pairs of different oligonucleotides may include sequences that are identical to overlapping portions of the predetermined nucleic acid fragment sequence as described herein (e.g., overlapping sequence portions from the same or from complementary strands of the nucleic acid fragment). In some embodiments, the plurality of oligonucleotides includes a set of oligonucleotides having sequences that combine to span the entire positive sequence and a set oligonucleotides having sequences that combine to span the entire negative sequence of the predetermined nucleic acid fragment. However, in certain embodiments, the plurality of oligonucleotides may include one or more oligonucleotides with sequences that are identical to sequence portions on one strand (either the positive or negative strand) of the nucleic acid fragment, but no oligonucleotides with sequences that are complementary to those sequence portions. In one embodiment, a plurality of oligonucleotides includes only oligonucleotides having sequences identical to portions of the positive sequence of the predetermined nucleic acid fragment. In one embodiment, a plurality of oligonucleotides includes only oligonucleotides having sequences identical to portions of the negative sequence of the predetermined nucleic acid fragment. These oligonucleotides may be assembled by sequential ligation or in an extension-based reaction (e.g., if an oligonucleotide having a 3′ region that is complementary to one of the plurality of oligonucleotides is added to the reaction).

In one aspect, a nucleic acid fragment may be assembled in a polymerase-mediated assembly reaction from a plurality of oligonucleotides that are combined and extended in one or more rounds of polymerase-mediated extensions. In another aspect, a nucleic acid fragment may be assembled in a ligase-mediated reaction from a plurality of oligonucleotides that are combined and ligated in one or more rounds of ligase-mediated ligations. In another aspect, a nucleic acid fragment may be assembled in a non-enzymatic reaction (e.g., a chemical reaction) from a plurality of oligonucleotides that are combined and assembled in one or more rounds of non-enzymatic reactions. In some embodiments, a nucleic acid fragment may be assembled using a combination of polymerase, ligase, and/or non-enzymatic reactions. For example, both polymerase(s) and ligase(s) may be included in an assembly reaction mixture. Accordingly, a nucleic acid may be assembled via coupled amplification and ligation or ligation during amplification. The resulting nucleic acid fragment from each assembly technique may have a sequence that includes the sequences of each of the plurality of assembly oligonucleotides that were used as described herein. These assembly reactions may be referred to as primerless assemblies, since the target nucleic acid is generated by assembling the input oligonucleotides rather than being generated in an amplification reaction where the oligonucleotides act as amplification primers to amplify a pre-existing template nucleic acid molecule corresponding to the target nucleic acid.

Polymerase-based assembly techniques may involve one or more suitable polymerase enzymes that can catalyze a template-based extension of a nucleic acid in a 5′ to 3′ direction in the presence of suitable nucleotides and an annealed template. A polymerase may be thermostable. A polymerase may be obtained from recombinant or natural sources. In some embodiments, a thermostable polymerase from a thermophilic organism may be used. In some embodiments, a polymerase may include a 3′→5′ exonuclease/proofreading activity. In some embodiments, a polymerase may have no, or little, proofreading activity (e.g., a polymerase may be a recombinant variant of a natural polymerase that has been modified to reduce its proofreading activity). Examples of thermostable DNA polymerases include, but are not limited to: Taq (a heat-stable DNA polymerase from the bacterium Thermus aquaticus); Pfu (a thermophilic DNA polymerase with a 3′→5′ exonuclease/proofreading activity from Pyrococcus furiosus, available from for example Promega); VentR® DNA Polymerase and Vent® (exo-) DNA Polymerase (thermophilic DNA polymerases with or without a 3′→5′ exonuclease/proofreading activity from Thermococcus litoralis; also known as Tli polymerase); Deep VentR® DNA Polymerase and Deep VentR® (exo-) DNA Polymerase (thermophilic DNA polymerases with or without a 3′ 5′ exonuclease/proofreading activity from Pyrococcus species GB-D; available from New England Biolabs); KOD HiFi (a recombinant Thermococcus kodakaraensis KOD1 DNA polymerase with a 3′→5′ exonuclease/proofreading activity, available from Novagen,); BIO-X-ACT (a mix of polymerases that possesses 5′-3′ DNA polymerase activity and 3′→5′ proofreading activity); Klenow Fragment (an N-terminal truncation of E. coli DNA Polymerase I which retains polymerase activity, but has lost the 5′→3′ exonuclease activity, available from, for example, Promega and NEB); Sequenase™ (T7 DNA polymerase deficient in 3′-5′ exonuclease activity); Phi29 (bacteriophage 29 DNA polymerase, may be used for rolling circle amplification, for example, in a TempliPhi™ DNA Sequencing Template Amplification Kit, available from Amersham Biosciences); TopoTaq™ (a hybrid polymerase that combines hyperstable DNA binding domains and the DNA unlinking activity of Methanopyrus topoisomerase, with no exonuclease activity, available from Fidelity Systems); TopoTaq HiFi which incorporates a proofreading domain with exonuclease activity; Phusion™ (a Pyrococcus-like enzyme with a processivity-enhancing domain, available from New England Biolabs); any other suitable DNA polymerase, or any combination of two or more thereof.

Ligase-based assembly techniques may involve one or more suitable ligase enzymes that can catalyze the covalent linking of adjacent 3′ and 5′ nucleic acid termini (e.g., a 5′ phosphate and a 3′ hydroxyl of nucleic acid(s) annealed on a complementary template nucleic acid such that the 3′ terminus is immediately adjacent to the 5′ terminus). Accordingly, a ligase may catalyze a ligation reaction between the 5′ phosphate of a first nucleic acid to the 3′ hydroxyl of a second nucleic acid if the first and second nucleic acids are annealed next to each other on a template nucleic acid). A ligase may be obtained from recombinant or natural sources. A ligase may be a heat-stable ligase. In some embodiments, a thermostable ligase from a thermophilic organism may be used. Examples of thermostable DNA ligases include, but are not limited to: Tth DNA ligase (from Thermus thermophilus, available from, for example, Eurogentec and GeneCraft); Pfu DNA ligase (a hyperthermophilic ligase from Pyrococcus furiosus); Taq ligase (from Thermus aquaticus), any other suitable heat-stable ligase, or any combination thereof. In some embodiments, one or more lower temperature ligases may be used (e.g., T4 DNA ligase). A lower temperature ligase may be useful for shorter overhangs (e.g., about 3, about 4, about 5, or about 6 base overhangs) that may not be stable at higher temperatures.

Non-enzymatic techniques can be used to ligate nucleic acids. For example, a 5′-end (e.g., the 5′ phosphate group) and a 3′-end (e.g., the 3′ hydroxyl) of one or more nucleic acids may be covalently linked together without using enzymes (e.g., without using a ligase). In some embodiments, non-enzymatic techniques may offer certain advantages over enzyme-based ligations. For example, non-enzymatic techniques may have a high tolerance of non-natural nucleotide analogues in nucleic acid substrates, may be used to ligate short nucleic acid substrates, may be used to ligate RNA substrates, and/or may be cheaper and/or more suited to certain automated (e.g., high throughput) applications.

Non-enzymatic ligation may involve a chemical ligation. In some embodiments, nucleic acid termini of two or more different nucleic acids may be chemically ligated. In some embodiments, nucleic acid termini of a single nucleic acid may be chemically ligated (e.g., to circularize the nucleic acid). It should be appreciated that both strands at a first double-stranded nucleic acid terminus may be chemically ligated to both strands at a second double-stranded nucleic acid terminus. However, in some embodiments only one strand of a first nucleic acid terminus may be chemically ligated to a single strand of a second nucleic acid terminus. For example, the 5′ end of one strand of a first nucleic acid terminus may be ligated to the 3′ end of one strand of a second nucleic acid terminus without the ends of the complementary strands being chemically ligated.

Accordingly, a chemical ligation may be used to form a covalent linkage between a 5′ terminus of a first nucleic acid end and a 3′ terminus of a second nucleic acid end, wherein the first and second nucleic acid ends may be ends of a single nucleic acid or ends of separate nucleic acids. In one aspect, chemical ligation may involve at least one nucleic acid substrate having a modified end (e.g., a modified 5′ and/or 3′ terminus) including one or more chemically reactive moieties that facilitate or promote linkage formation. In some embodiments, chemical ligation occurs when one or more nucleic acid termini are brought together in close proximity (e.g., when the termini are brought together due to annealing between complementary nucleic acid sequences). Accordingly, annealing between complementary 3′ or 5′ overhangs (e.g., overhangs generated by restriction enzyme cleavage of a double-stranded nucleic acid) or between any combination of complementary nucleic acids that results in a 3′ terminus being brought into close proximity with a 5′ terminus (e.g., the 3′ and 5′ termini are adjacent to each other when the nucleic acids are annealed to a complementary template nucleic acid) may promote a template-directed chemical ligation. Examples of chemical reactions may include, but are not limited to, condensation, reduction, and/or photo-chemical ligation reactions. It should be appreciated that in some embodiments chemical ligation can be used to produce naturally-occurring phosphodiester internucleotide linkages, non-naturally-occurring phosphamide pyrophosphate internucleotide linkages, and/or other non-naturally-occurring internucleotide linkages.

In some embodiments, the process of chemical ligation may involve one or more coupling agents to catalyze the ligation reaction. A coupling agent may promote a ligation reaction between reactive groups in adjacent nucleic acids (e.g., between a 5′-reactive moiety and a 3′-reactive moiety at adjacent sites along a complementary template). In some embodiments, a coupling agent may be a reducing reagent (e.g., ferricyanide), a condensing reagent such (e.g., cyanoimidazole, cyanogen bromide, carbodiimide, etc.), or irradiation (e.g., UV irradiation for photo-ligation).

In some embodiments, a chemical ligation may be an autoligation reaction that does not involve a separate coupling agent. In autoligation, the presence of a reactive group on one or more nucleic acids may be sufficient to catalyze a chemical ligation between nucleic acid termini without the addition of a coupling agent (see, for example, Xu Y & Kool E T, 1997, Tetrahedron Lett. 38:5595-8). Non-limiting examples of these reagent-free ligation reactions may involve nucleophilic displacements of sulfur on bromoacetyl, tosyl, or iodo-nucleoside groups (see, for example, Xu Y et al., 2001, Nat Biotech 19:148-52). Nucleic acids containing reactive groups suitable for autoligation can be prepared directly on automated synthesizers (see, for example, Xu Y & Kool E T, 1999, Nuc. Acids Res. 27:875-81). In some embodiments, a phosphorothioate at a 3′ terminus may react with a leaving group (such as tosylate or iodide) on a thymidine at an adjacent 5′ terminus. In some embodiments, two nucleic acid strands bound at adjacent sites on a complementary target strand may undergo auto-ligation by displacement of a 5′-end iodide moiety (or tosylate) with a 3′-end sulfur moiety. Accordingly, in some embodiments the product of an autoligation may include a non-naturally-occurring internucleotide linkage (e.g., a single oxygen atom may be replaced with a sulfur atom in the ligated product).

In some embodiments, a synthetic nucleic acid duplex can be assembled via chemical ligation in a one step reaction involving simultaneous chemical ligation of nucleic acids on both strands of the duplex. For example, a mixture of 5′-phosphorylated oligonucleotides corresponding to both strands of a target nucleic acid may be chemically ligated by a) exposure to heat (e.g., to 97° C.) and slow cooling to form a complex of annealed oligonucleotides, and b) exposure to cyanogen bromide or any other suitable coupling agent under conditions sufficient to chemically ligate adjacent 3′ and 5′ ends in the nucleic acid complex.

In some embodiments, a synthetic nucleic acid duplex can be assembled via chemical ligation in a two step reaction involving separate chemical ligations for the complementary strands of the duplex. For example, each strand of a target nucleic acid may be ligated in a separate reaction containing phosphorylated oligonucleotides corresponding to the strand that is to be ligated and non-phosphorylated oligonucleotides corresponding to the complementary strand. The non-phosphorylated oligonucleotides may serve as a template for the phosphorylated oligonucleotides during a chemical ligation (e.g. using cyanogen bromide). The resulting single-stranded ligated nucleic acid may be purified and annealed to a complementary ligated single-stranded nucleic acid to form the target duplex nucleic acid (see, for example, Shabarova Z A et al., 1991, Nuc. Acids Res. 19:4247-51).

Aspects of the invention may be used to enhance different types of nucleic acid assembly reactions (e.g., multiplex nucleic acid assembly reactions). Aspects of the invention may be used in combination with one or more assembly reactions described in, for example, Carr et al., 2004, Nucleic Acids Research, Vol. 32, No 20, e162:9); Richmond et al., 2004, Nucleic Acids Research, Vol. 32, No 17, pp. 5011-5018; Caruthers et al., 1972, J. Mol. Biol. 72, 475-492; Hecker et al., 1998, Biotechniques 24:256-260; Kodumal et al., 2004, PNAS Vol. 101, No. 44, pp. 15573-15578; Tian et al., 2004, Nature, Vol. 432, pp. 1050-1054; and U.S. Pat. Nos. 6,008,031 and 5,922,539, the disclosures of which are incorporated herein by reference. Certain embodiments of multiplex nucleic acid assembly reactions for generating a predetermined nucleic acid fragment are illustrated with reference to FIGS. 1-4. It should be appreciated that synthesis and assembly methods described herein (including, for example, oligonucleotide synthesis, multiplex nucleic acid assembly, concerted assembly of nucleic acid fragments, or any combination thereof) may be performed in any suitable format, including in a reaction tube, in a multi-well plate, on a surface, on a column, in a microfluidic device (e.g., a microfluidic tube), a capillary tube, etc.

It should be appreciated that the reference to complementary nucleic acids or complementary nucleic acid regions herein refers to nucleic acids or regions thereof that have sequences which are reverse complements of each other so that they can hybridize in an antiparallel fashion typical of natural DNA.

FIG. 1 shows one embodiment of a plurality of oligonucleotides that may be assembled in a polymerase-based multiplex oligonucleotide assembly reaction. FIG. 1A shows two groups of oligonucleotides (Group P and Group N) that have sequences of portions of the two complementary strands of a nucleic acid fragment to be assembled. Group P includes oligonucleotides with positive strand sequences (P₁, P₂, . . . Pₙ₋₁, Pₙ, Pₙ₊₁, . . . P_{T}, shown from 5′→3′ on the positive strand). Group N includes oligonucleotides with negative strand sequences (N_{T}, . . . , Nₙ₊₁, Nₙ, Nₙ₋₁, . . . , N₂, N₁, shown from 5′→3′ on the negative strand). In this example, none of the P group oligonucleotides overlap with each other and none of the N group oligonucleotides overlap with each other. However, in some embodiments, one or more of the oligonucleotides within the S or N group may overlap. Furthermore, FIG. 1A shows gaps between consecutive oligonucleotides in Group P and gaps between consecutive oligonucleotides in Group N. However, each P group oligonucleotide (except for P₁) and each N group oligonucleotide (except for N_{T}) overlaps with complementary regions of two oligonucleotides from the complementary group of oligonucleotides. P₁ and N_{T} overlap with a complementary region of only one oligonucleotide from the other group (the complementary 3′-most oligonucleotides N₁ and P_{T}, respectively). FIG. 1B shows a structure of an embodiment of a Group P or Group N oligonucleotide represented in FIG. 1A. This oligonucleotide includes a 5′ region that is complementary to a 5′ region of a first oligonucleotide from the other group, a 3′ region that is complementary to a 3′ region of a second oligonucleotide from the other group, and a core or central region that is not complementary to any oligonucleotide sequence from the other group (or its own group). This central region is illustrated as the B region in FIG. 1B. The sequence of the B region may be different for each different oligonucleotide. As defined herein, the B region of an oligonucleotide in one group corresponds to a gap between two consecutive oligonucleotides in the complementary group of oligonucleotides. It should be noted that the 5′-most oligonucleotide in each group (P₁ in Group P and N_{T} in Group N) does not have a 5′ region that is complementary to the 5′ region of any other oligonucleotide in either group. Accordingly, the 5′-most oligonucleotides (P₁ and N_{T}) that are illustrated in FIG. 1A each have a 3′ complementary region and a 5′ non-complementary region (the B region of FIG. 1B), but no 5′ complementary region. However, it should be appreciated that any one or more of the oligonucleotides in Group P and/or Group N (including all of the oligonucleotides in Group P and/or Group N) can be designed to have no B region. In the absence of a B region, a 5′-most oligonucleotide has only the 3′ complementary region (meaning that the entire oligonucleotide is complementary to the 3′ region of the 3′-most oligonucleotide from the other group (e.g., the 3′ region of N₁ or P_{T} shown in FIG. 1A). In the absence of a B region, one of the other oligonucleotides in either Group P or Group N has only a 5′ complementary region and a 3′ complementary region (meaning that the entire oligonucleotide is complementary to the 5′ and 3′ sequence regions of the two overlapping oligonucleotides from the complementary group). In some embodiments, only a subset of oligonucleotides in an assembly reaction may include B regions. It should be appreciated that the length of the 5′, 3′, and B regions may be different for each oligonucleotide. However, for each oligonucleotide the length of the 5′ region is the same as the length of the complementary 5′ region in the 5′ overlapping oligonucleotide from the other group. Similarly, the length of the 3′ region is the same as the length of the complementary 3′ region in the 3′ overlapping oligonucleotide from the other group. However, in certain embodiments a 3′-most oligonucleotide may be designed with a 3′ region that extends beyond the 5′ region of the 5′-most oligonucleotide. In this embodiment, an assembled product may include the 5′ end of the 5′-most oligonucleotide, but not the 3′ end of the 3′-most oligonucleotide that extends beyond it.

FIG. 1C illustrates a subset of the oligonucleotides from FIG. 1A, each oligonucleotide having a 5′, a 3′, and an optional B region. Oligonucleotide Pₙ is shown with a 5′ region that is complementary to (and can anneal to) the 5′ region of oligonucleotide Nₙ₋₁. Oligonucleotide Pₙ also has a 3′ region that is complementary to (and can anneal to) the 3′ region of oligonucleotide Nₙ. Nₙ is also shown with a 5′ region that is complementary (and can anneal to) the 5′ region of oligonucleotide Pₙ₊₁. This pattern could be repeated for all of oligonucleotides P₂ to P_{T} and N₁ to N_{T-1} (with the 5′-most oligonucleotides only having 3′ complementary regions as discussed herein). If all of the oligonucleotides from Group P and Group N are mixed together under appropriate hybridization conditions, they may anneal to form a long chain such as the oligonucleotide complex illustrated in FIG. 1A. However, subsets of the oligonucleotides may form shorter chains and even oligonucleotide dimers with annealed 5′ or 3′ regions. It should be appreciated that many copies of each oligonucleotide are included in a typical reaction mixture. Accordingly, the resulting hybridized reaction mixture may contain a distribution of different oligonucleotide dimers and complexes. Polymerase-mediated extension of the hybridized oligonucleotides results in a template-based extension of the 3′ ends of oligonucleotides that have annealed 3′ regions. Accordingly, polymerase-mediated extension of the oligonucleotides shown in FIG. 1C would result in extension of the 3′ ends only of oligonucleotides Pₙ and Nₙ generating extended oligonucleotides containing sequences that are complementary to all the regions of Nₙ and Pₙ, respectively. Extended oligonucleotide products with sequences complementary to all of Nₙ₋₁ and Pₙ₊₁ would not be generated unless oligonucleotides Pₙ₋₁ and Nₙ₊₁ were included in the reaction mixture. Accordingly, if all of the oligonucleotide sequences in a plurality of oligonucleotides are to be incorporated into an assembled nucleic acid fragment using a polymerase, the plurality of oligonucleotides should include 5′-most oligonucleotides that are at least complementary to the entire 3′ regions of the 3′-most oligonucleotides. In some embodiments, the 5′-most oligonucleotides also may have 5′ regions that extend beyond the 3′ ends of the 3′-most oligonucleotides as illustrated in FIG. 1A. In some embodiments, a ligase also may be added to ligate adjacent 5′ and 3′ ends that may be formed upon 3′ extension of annealed oligonucleotides in an oligonucleotide complex such as the one illustrated in FIG. 1A.

When assembling a nucleic acid fragment using a polymerase, a single cycle of polymerase extension extends oligonucleotide pairs with annealed 3′ regions. Accordingly, if a plurality of oligonucleotides were annealed to form an annealed Pₙ₊₁ are complementary, and the 3′ regions of Pₙ₊₁ and Nₙ₊₁ are complementary. It also should be appreciated that the reaction products shown in FIG. 1D are a subset of the reaction products that would be obtained using all of the oligonucleotides of Group P and Group N. A first polymerase extension reaction using all of the oligonucleotides would result in a plurality of overlapping oligonucleotide dimers from P₁/N₁ to P_{T}/N_{T}. Each of these may be denatured and at least one of the strands could then anneal to an overlapping complementary strand from an adjacent (either 3′ or 5′) oligonucleotide dimer and be extended in a second cycle of polymerase extension as shown in FIG. 1D. Subsequent cycles of denaturing, annealing, and extension produce progressively larger products including a nucleic acid fragment that includes the sequences of all of the initial oligonucleotides. It should be appreciated that these subsequent rounds of extension also produce many nucleic acid products of intermediate length. The reaction product may be complex since not all of the 3′ regions may be extended in each cycle. Accordingly, unextended oligonucleotides may be available in each cycle to anneal to other unextended oligonucleotides or to previously extended oligonucleotides. Similarly, extended products of different sizes may anneal to each other in each cycle. Accordingly, a mixture of extended products of different sizes covering different regions of the sequence may be generated along with the nucleic acid fragment covering the entire sequence. This mixture also may contain any remaining unextended oligonucleotides.

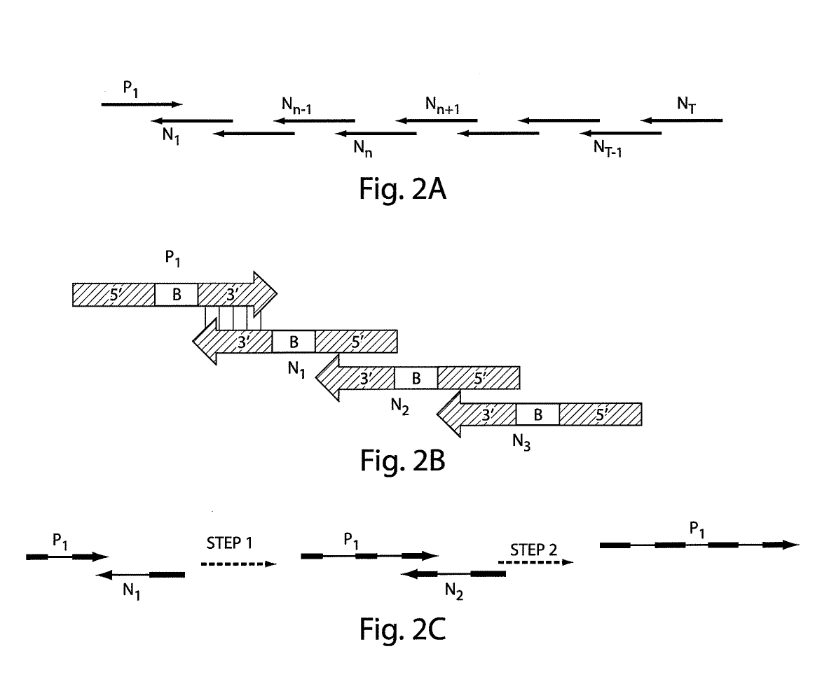

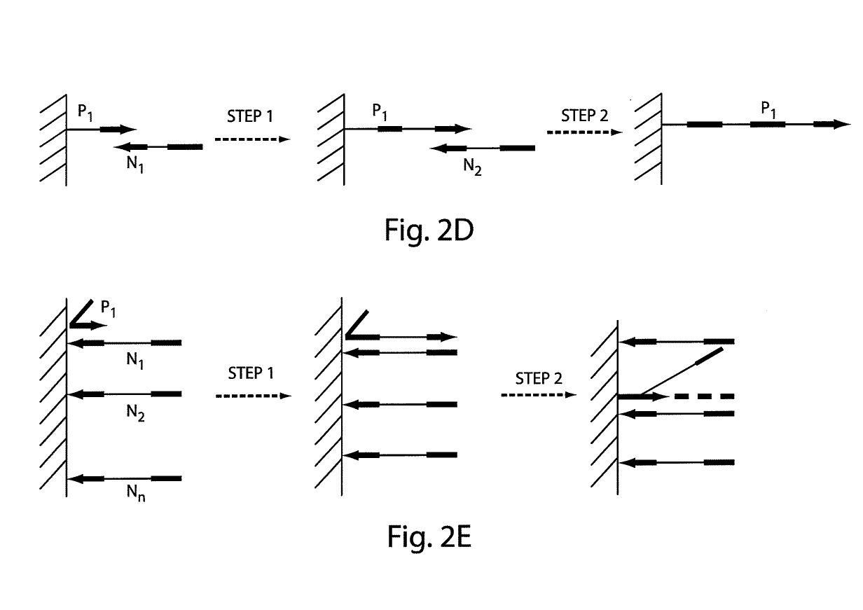

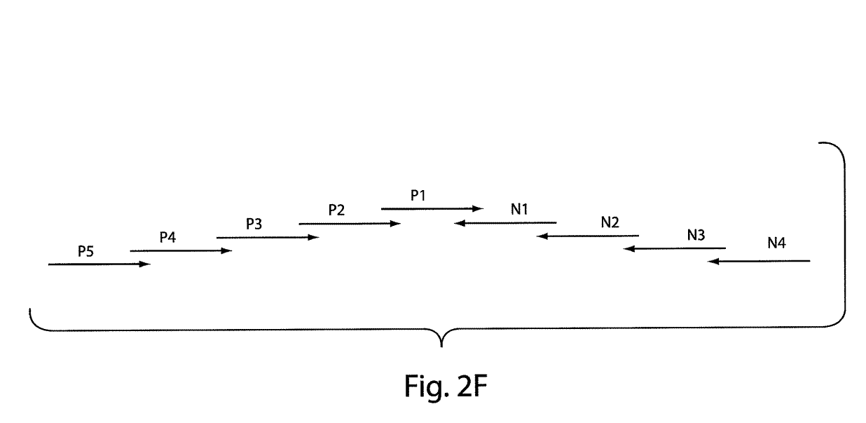

FIG. 2 shows an embodiment of a plurality of oligonucleotides that may be assembled in a directional polymerase-based multiplex oligonucleotide assembly reaction. In this embodiment, only the 5′-most oligonucleotide of Group P may be provided. In contrast to the example shown in FIG. 1, the remainder of the sequence of the predetermined nucleic acid fragment is provided by oligonucleotides of Group N. The 3′-most oligonucleotide of Group N (N1) has a 3′ region that is complementary to the 3′ region of P₁ as shown in FIG. 2B. However, the remainder of the oligonucleotides in Group N have overlapping (but non-complementary) 3′ and 5′ regions as illustrated in FIG. 2B for oligonucleotides N1-N3. Each Group N oligonucleotide (e.g., Nₙ) overlaps with two adjacent oligonucleotides: one overlaps with the 3′ region (Nₙ₋₁) and one with the 5′ region (Nₙ₊₁) except for N₁ that overlaps with the 3′ regions of P₁ (complementary overlap) and N2 (non-complementary overlap), and NT that overlaps only with N_{T-1}. It complex such as the one illustrated in FIG. 1A, a single cycle of polymerase extension would result in the extension of the 3′ ends of the P₁/N₁, P₂/N₂, . . . , P_{n'1}/Nₙ₋₁, Pₙ/Nₙ, Pₙ₊₁/Nₙ₊₁, . . . , P_{T}/N_{T} oligonucleotide pairs. In one embodiment, a single molecule could be generated by ligating the extended oligonucleotide dimers. In one embodiment, a single molecule incorporating all of the oligonucleotide sequences may be generated by performing several polymerase extension cycles.

In one embodiment, FIG. 1D illustrates two cycles of polymerase extension (separated by a denaturing step and an annealing step) and the resulting nucleic acid products. It should be appreciated that several cycles of polymerase extension may be required to assemble a single nucleic acid fragment containing all the sequences of an initial plurality of oligonucleotides. In one embodiment, a minimal number of extension cycles for assembling a nucleic acid may be calculated as log₂n, where n is the number of oligonucleotides being assembled. In some embodiments, progressive assembly of the nucleic acid may be achieved without using temperature cycles. For example, an enzyme capable of rolling circle amplification may be used (e.g., phi 29 polymerase) when a circularized nucleic acid (e.g., oligonucleotide) complex is used as a template to produce a large amount of circular product for subsequent processing using MutS or a MutS homolog as described herein. In step 1 of FIG. 1D, annealed oligonucleotide pairs Pₙ/Nₙ and Pₙ₊₁/Nₙ₊₁ are extended to form oligonucleotide dimer products incorporating the sequences covered by the respective oligonucleotide pairs. For example, Pₙ is extended to incorporate sequences that are complementary to the B and 5′ regions of Nₙ (indicated as N′ₙ in FIG. 1D). Similarly, Nₙ₊₁ is extended to incorporate sequences that are complementary to the 5′ and B regions of Pₙ₊₁ (indicated as P′ₙ₊₁ in FIG. 1D). These dimer products may be denatured and reannealed to form the starting material of step 2 where the 3′ end of the extended Pₙ₊₁ oligonucleotide is annealed to the 3′ end of the extended Nₙ₊₁ oligonucleotide. This product may be extended in a polymerase-mediated reaction to form a product that incorporates the sequences of the four oligonucleotides (Pₙ, Nₙ, Pₙ+1, Nₙ₊₁). One strand of this extended product has a sequence that includes (in 5′ to 3′ order) the 5′, B, and 3′ regions of Pₙ, the complement of the B region of Nₙ, the 5′, B, and 3′ regions of Pₙ₊₁, and the complements of the B and 5′ regions of Nₙ₊₁. The other strand of this extended product has the complementary sequence. It should be appreciated that the 3′ regions of Pₙ and Nₙ are complementary, the 5′ regions of Nₙ and should be appreciated that all of the overlaps shown in FIG. 2A between adjacent oligonucleotides N₂ to N_{T-1} are non-complementary overlaps between the 5′ region of one oligonucleotide and the 3′ region of the adjacent oligonucleotide illustrated in a 3′ to 5′ direction on the N strand of the predetermined nucleic acid fragment. It also should be appreciated that each oligonucleotide may have 3′, B, and 5′regions of different lengths (including no B region in some embodiments). In some embodiments, none of the oligonucleotides may have B regions, meaning that the entire sequence of each oligonucleotide may overlap with the combined 5′ and 3′ region sequences of its two adjacent oligonucleotides.

Assembly of a predetermined nucleic acid fragment from the plurality of oligonucleotides shown in FIG. 2A may involve multiple cycles of polymerase-mediated extension. Each extension cycle may be separated by a denaturing and an annealing step.

FIG. 2C illustrates the first two steps in this assembly process. In step 1, annealed oligonucleotides P₁ and N₁ are extended to form an oligonucleotide dimer. P₁ is shown with a 5′ region that is non-complementary to the 3′ region of N₁ and extends beyond the 3′ region of N₁ when the oligonucleotides are annealed. However, in some embodiments, P₁ may lack the 5′ non-complementary region and include only sequences that overlap with the 3′ region of N₁. The product of P₁ extension is shown after step 1 containing an extended region that is complementary to the 5′ end of N₁. The single strand illustrated in FIG. 2C may be obtained by denaturing the oligonucleotide dimer that results from the extension of P₁/N₁ in step 1. The product of P₁ extension is shown annealed to the 3′ region of N₂. This annealed complex may be extended in step 2 to generate an extended product that now includes sequences complementary to the B and 5′ regions of N₂. Again, the single strand illustrated in FIG. 2C may be obtained by denaturing the oligonucleotide dimer that results from the extension reaction of step 2. Additional cycles of extension may be performed to further assemble a predetermined nucleic acid fragment. In each cycle, extension results in the addition of sequences complementary to the B and 5′ regions of the next Group N oligonucleotide. Each cycle may include a denaturing and annealing step. However, the extension may occur under the annealing conditions. Accordingly, in one embodiment, cycles of extension may be obtained by alternating between denaturing conditions (e.g., a denaturing temperature) and annealing/extension conditions (e.g., an annealing/extension temperature). In one embodiment, T (the number of group N oligonucleotides) may determine the minimal number of temperature cycles used to assemble the oligonucleotides. However, in some embodiments, progressive extension may be achieved without temperature cycling. For example, an enzyme capable promoting rolling circle amplification may be used (e.g., TempliPhi). It should be appreciated that a reaction mixture containing an assembled predetermined nucleic acid fragment also may contain a distribution of shorter extension products that may result from incomplete extension during one or more of the cycles or may be the result of an P₁/N₁ extension that was initiated after the first cycle.

FIG. 2D illustrates an example of a sequential extension reaction where the 5′-most P₁ oligonucleotide is bound to a support and the Group N oligonucleotides are unbound. The reaction steps are similar to those described for FIG. 2C. However, an extended predetermined nucleic acid fragment will be bound to the support via the 5′-most P₁ oligonucleotide. Accordingly, the complementary strand (the negative strand) may readily be obtained by denaturing the bound fragment and releasing the negative strand. In some embodiments, the attachment to the support may be labile or readily reversed (e.g., using light, a chemical reagent, a pH change, etc.) and the positive strand also may be released. Accordingly, either the positive strand, the negative strand, or the double-stranded product may be obtained. FIG. 2E illustrates an example of a sequential reaction where P₁ is unbound and the Group N oligonucleotides are bound to a support. The reaction steps are similar to those described for FIG. 2C. However, an extended predetermined nucleic acid fragment will be bound to the support via the 5′-most N_{T} oligonucleotide. Accordingly, the complementary strand (the positive strand) may readily be obtained by denaturing the bound fragment and releasing the positive strand. In some embodiments, the attachment to the support may be labile or readily reversed (e.g., using light, a chemical reagent, a pH change, etc.) and the negative strand also may be released. Accordingly, either the positive strand, the negative strand, or the double-stranded product may be obtained.

It should be appreciated that other configurations of oligonucleotides may be used to assemble a nucleic acid via two or more cycles of polymerase-based extension. In many configurations, at least one pair of oligonucleotides have complementary 3′ end regions. FIG. 2F illustrates an example where an oligonucleotide pair with complementary 3′ end regions is flanked on either side by a series of oligonucleotides with overlapping non-complementary sequences. The oligonucleotides illustrated to the right of the complementary pair have overlapping 3′ and 5′ regions (with the 3′ region of one oligonucleotide being identical to the 5′ region of the adjacent oligonucleotide) that corresponding to a sequence of one strand of the target nucleic acid to be assembled. The oligonucleotides illustrated to the left of the complementary pair have overlapping 3′ and 5′ regions (with the 3′ region of one oligonucleotide being identical to the 5′ region of the adjacent oligonucleotide) that correspond to a sequence of the complementary strand of the target nucleic acid. These oligonucleotides may be assembled via sequential polymerase-based extension reactions as described herein (see also, for example, Xiong et al., 2004, Nucleic Acids Research, Vol. 32, No. 12, e98:10, the disclosure of which is incorporated by reference herein). It should be appreciated that different numbers and/or lengths of oligonucleotides may be used on either side of the complementary pair. Accordingly, the illustration of the complementary pair as the central pair in FIG. 2F is not intended to be limiting as other configuration of a complementary oligonucleotide pair flanked by a different number of non-complementary pairs on either side may be used according to methods of the invention.

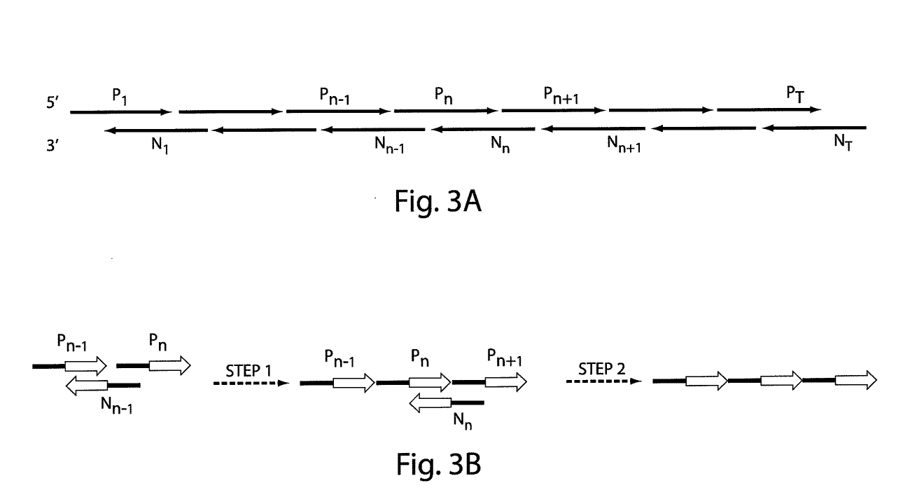

FIG. 3 shows an embodiment of a plurality of oligonucleotides that may be assembled in a ligase reaction. FIG. 3A illustrates the alignment of the oligonucleotides showing that they do not contain gaps (i.e., no B region as described herein). Accordingly, the oligonucleotides may anneal to form a complex with no nucleotide gaps between the 3′ and 5′ ends of the annealed oligonucleotides in either Group P or Group N. These oligonucleotides provide a suitable template for assembly using a ligase under appropriate reaction conditions. However, it should be appreciated that these oligonucleotides also may be assembled using a polymerase-based assembly reaction as described herein. FIG. 3B shows two individual ligation reactions. These reactions are illustrated in two steps. However, it should be appreciated that these ligation reactions may occur simultaneously or sequentially in any order and may occur as such in a reaction maintained under constant reaction conditions (e.g., with no temperature cycling) or in a reaction exposed to several temperature cycles. For example, the reaction illustrated in step 2 may occur before the reaction illustrated in step 1. In each ligation reaction illustrated in FIG. 3B, a Group N oligonucleotide is annealed to two adjacent Group P oligonucleotides (due to the complementary 5′ and 3′ regions between the P and N oligonucleotides), providing a template for ligation of the adjacent P oligonucleotides. Although not illustrated, ligation of the N group oligonucleotides also may proceed in similar manner to assemble adjacent N oligonucleotides that are annealed to their complementary P oligonucleotide. Assembly of the predetermined nucleic acid fragment may be obtained through ligation of all of the oligonucleotides to generate a double stranded product. However, in some embodiments, a single stranded product of either the positive or negative strand may be obtained. In certain embodiments, a plurality of oligonucleotides may be designed to generate only single-stranded reaction products in a ligation reaction. For example, a first group of oligonucleotides (of either Group P or Group N) may be provided to cover the entire sequence on one strand of the predetermined nucleic acid fragment (on either the positive or negative strand). In contrast, a second group of oligonucleotides (from the complementary group to the first group) may be designed to be long enough to anneal to complementary regions in the first group but not long enough to provide adjacent 5′ and 3′ ends between oligonucleotides in the second group. This provides substrates that are suitable for ligation of oligonucleotides from the first group but not the second group. The result is a single-stranded product having a sequence corresponding to the oligonucleotides in the first group. Again, as with other assembly reactions described herein, a ligase reaction mixture that contains an assembled predetermined nucleic acid fragment also may contain a distribution of smaller fragments resulting from the assembly of a subset of the oligonucleotides.

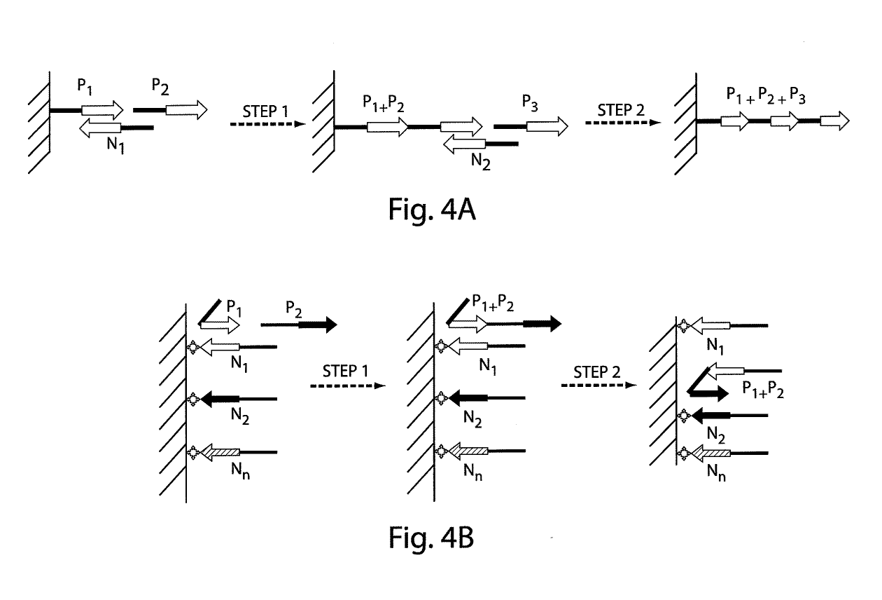

FIG. 4 shows an embodiment of a ligase-based assembly where one or more of the plurality of oligonucleotides is bound to a support. In FIG. 4A, the 5′ most oligonucleotide of the P group oligonucleotides is bound to a support. Ligation of adjacent oligonucleotides in the 5′ to 3′ direction results in the assembly of a predetermined nucleic acid fragment. FIG. 4A illustrates an example where adjacent oligonucleotides P₂ and P₃ are added sequentially. However, the ligation of any two adjacent oligonucleotides from Group P may occur independently and in any order in a ligation reaction mixture. For example, when P₁ is ligated to the 5′ end of N₂, N₂ may be in the form of a single oligonucleotide or it already may be ligated to one or more downstream oligonucleotides (N3, N₄, etc.). It should be appreciated that for a ligation assembly bound to a support, either the 5′-most (e.g., P₁ for Group P, or N_{T} for Group N) or the 3′-most (e.g., P_{T} for Group P, or N₁ for Group N) oligonucleotide may be bound to a support since the reaction can proceed in any direction. In some embodiments, a predetermined nucleic acid fragment may be assembled with a central oligonucleotide (i.e., neither the 5′-most or the 3′-most) that is bound to a support provided that the attachment to the support does not interfere with ligation.

FIG. 4B illustrates an example where a plurality of N group oligonucleotides are bound to a support and a predetermined nucleic acid fragment is assembled from P group oligonucleotides that anneal to their complementary support-bound N group oligonucleotides. Again, FIG. 4B illustrates a sequential addition. However, adjacent P group oligonucleotides may be ligated in any order. Also, the bound oligonucleotides may be attached at their 5′ end, 3′ end, or at any other position provided that the attachment does not interfere with their ability to bind to complementary 5′ and 3′ regions on the oligonucleotides that are being assembled. This reaction may involve one or more reaction condition changes (e.g., temperature cycles) so that ligated oligonucleotides bound to one immobilized N group oligonucleotide can be dissociated from the support and bind to a different immobilized N group oligonucleotide to provide a substrate for ligation to another P group oligonucleotide.

As with other assembly reactions described herein, support-bound ligase reactions (e.g., those illustrated in FIG. 4B) that generate a full length predetermined nucleic acid fragment also may generate a distribution of smaller fragments resulting from the assembly of subsets of the oligonucleotides. A support used in any of the assembly reactions described herein (e.g., polymerase-based, ligase-based, or other assembly reaction) may include any suitable support medium. A support may be solid, porous, a matrix, a gel, beads, beads in a gel, etc. A support may be of any suitable size. A solid support may be provided in any suitable configuration or shape (e.g., a chip, a bead, a gel, a microfluidic channel, a planar surface, a spherical shape, a column, etc.).

As illustrated herein, different oligonucleotide assembly reactions may be used to assemble a plurality of overlapping oligonucleotides (with overlaps that are either 5′/5′, 3′/3′, 5′/3′, complementary, non-complementary, or a combination thereof). Many of these reactions include at least one pair of oligonucleotides (the pair including one oligonucleotide from a first group or P group of oligonucleotides and one oligonucleotide from a second group or N group of oligonucleotides) have overlapping complementary 3′ regions. However, in some embodiments, a predetermined nucleic acid may be assembled from non-overlapping oligonucleotides using blunt-ended ligation reactions. In some embodiments, the order of assembly of the non-overlapping oligonucleotides may be biased by selective phosphorylation of different 5′ ends. In some embodiments, size purification may be used to select for the correct order of assembly. In some embodiments, the correct order of assembly may be promoted by sequentially adding appropriate oligonucleotide substrates into the reaction (e.g., the ligation reaction).

In order to obtain a full-length nucleic acid fragment from a multiplex oligonucleotide assembly reaction, a purification step may be used to remove starting oligonucleotides and/or incompletely assembled fragments. In some embodiments, a purification step may involve chromatography, electrophoresis, or other physical size separation technique. In certain embodiments, a purification step may involve amplifying the full length product. For example, a pair of amplification primers (e.g., PCR primers) that correspond to the predetermined 5′ and 3′ ends of the nucleic acid fragment being assembled will preferentially amplify full length product in an exponential fashion. It should be appreciated that smaller assembled products may be amplified if they contain the predetermined 5′ and 3′ ends. However, such smaller-than-expected products containing the predetermined 5′ and 3′ ends should only be generated if an error occurred during assembly (e.g., resulting in the deletion or omission of one or more regions of the target nucleic acid) and may be removed by size fractionation of the amplified product. Accordingly, a preparation containing a relatively high amount of full length product may be obtained directly by amplifying the product of an assembly reaction using primers that correspond to the predetermined 5′ and 3′ ends. In some embodiments, additional purification (e.g., size selection) techniques may be used to obtain a more purified preparation of amplified full-length nucleic acid fragment.

When designing a plurality of oligonucleotides to assemble a predetermined nucleic acid fragment, the sequence of the predetermined fragment will be provided by the oligonucleotides as described herein. However, the oligonucleotides may contain additional sequence information that may be removed during assembly or may be provided to assist in subsequent manipulations of the assembled nucleic acid fragment. Examples of additional sequences include, but are not limited to, primer recognition sequences for amplification (e.g., PCR primer recognition sequences), restriction enzyme recognition sequences, recombination sequences, other binding or recognition sequences, labeled sequences, etc. In some embodiments, one or more of the 5′-most oligonucleotides, one or more of the 3′-most oligonucleotides, or any combination thereof, may contain one or more additional sequences. In some embodiments, the additional sequence information may be contained in two or more adjacent oligonucleotides on either strand of the predetermined nucleic acid sequence. Accordingly, an assembled nucleic acid fragment may contain additional sequences that may be used to connect the assembled fragment to one or more additional nucleic acid fragments (e.g., one or more other assembled fragments, fragments obtained from other sources, vectors, etc.) via ligation, recombination, polymerase-mediated assembly, etc. In some embodiments, purification may involve cloning one or more assembled nucleic acid fragments. The cloned product may be screened (e.g., sequenced, analyzed for an insert of the expected size, etc.).

In some embodiments, a nucleic acid fragment assembled from a plurality of oligonucleotides may be combined with one or more additional nucleic acid fragments using a polymerase-based and/or a ligase-based extension reaction similar to those described herein for oligonucleotide assembly. Accordingly, one or more overlapping nucleic acid fragments may be combined and assembled to produce a larger nucleic acid fragment as described herein. In certain embodiments, double-stranded overlapping oligonucleotide fragments may be combined. However, single-stranded fragments, or combinations of single-stranded and double-stranded fragments may be combined as described herein. A nucleic acid fragment assembled from a plurality of oligonucleotides may be of any length depending on the number and length of the oligonucleotides used in the assembly reaction. For example, a nucleic acid fragment (either single-stranded or double-stranded) assembled from a plurality of oligonucleotides may be between 50 and 1,000 nucleotides long (for example, about 70 nucleotides long, between 100 and 500 nucleotides long, between 200 and 400 nucleotides long, about 200 nucleotides long, about 300 nucleotides long, about 400 nucleotides long, etc.). One or more such nucleic acid fragments (e.g., with overlapping 3′ and/or 5′ ends) may be assembled to form a larger nucleic acid fragment (single-stranded or double-stranded) as described herein.

A full length product assembled from smaller nucleic acid fragments also may be isolated or purified as described herein (e.g., using a size selection, cloning, selective binding or other suitable purification procedure). In addition, any assembled nucleic acid fragment (e.g., full-length nucleic acid fragment) described herein may be amplified (prior to, as part of, or after, a purification procedure) using appropriate 5′ and 3′ amplification primers.

### Synthetic Oligonucleotides

It should be appreciated that the terms P Group and N Group oligonucleotides are used herein for clarity purposes only, and to illustrate several embodiments of multiplex oligonucleotide assembly. The Group P and Group N oligonucleotides described herein are interchangeable, and may be referred to as first and second groups of oligonucleotides corresponding to sequences on complementary strands of a target nucleic acid fragment.

Oligonucleotides may be synthesized using any suitable technique. For example, oligonucleotides may be synthesized on a column or other support (e.g., a chip). Examples of chip-based synthesis techniques include techniques used in synthesis devices or methods available from Combimatrix, Agilent, Affymetrix, or other sources. A synthetic oligonucleotide may be of any suitable size, for example between 10 and 1,000 nucleotides long (e.g., between 10 and 200, 200 and 500, 500 and 1,000 nucleotides long, or any combination thereof). An assembly reaction may include a plurality of oligonucleotides, each of which independently may be between 10 and 200 nucleotides in length (e.g., between 20 and 150, between 30 and 100, 30 to 90, 30-80, 30-70, 30-60, 35-55, 40-50, or any intermediate number of nucleotides). However, one or more shorter or longer oligonucleotides may be used in certain embodiments.

Oligonucleotides may be provided as single stranded synthetic products. However, in some embodiments, oligonucleotides may be provided as double-stranded preparations including an annealed complementary strand. Oligonucleotides may be molecules of DNA, RNA, PNA, or any combination thereof. A double-stranded oligonucleotide may be produced by amplifying a single-stranded synthetic oligonucleotide or other suitable template (e.g., a sequence in a nucleic acid preparation such as a nucleic acid vector or genomic nucleic acid). Accordingly, a plurality of oligonucleotides designed to have the sequence features described herein may be provided as a plurality of single-stranded oligonucleotides having those feature, or also may be provided along with complementary oligonucleotides. In some embodiments, an oligonucleotide may be phosphorylated (e.g., with a 5′ phosphate). In some embodiments, an oligonucleotide may be non-phosphorylated.

In some embodiments, an oligonucleotide may be amplified using an appropriate primer pair with one primer corresponding to each end of the oligonucleotide (e.g., one that is complementary to the 3′ end of the oligonucleotide and one that is identical to the 5′ end of the oligonucleotide). In some embodiments, an oligonucleotide may be designed to contain a central assembly sequence (corresponding to a target sequence, designed to be incorporated into the final product) flanked by a 5′ amplification sequence (e.g., a 5′ universal sequence) and a 3′ amplification sequence (e.g., a 3′ universal sequence). Amplification primers (e.g., between 10 and 50 nucleotides long, between 15 and 45 nucleotides long, about 25 nucleotides long, etc.) corresponding to the flanking amplification sequences may be used to amplify the oligonucleotide (e.g., one primer may be complementary to the 3′ amplification sequence and one primer may have the same sequence as the 5′ amplification sequence). The amplification sequences then may be removed from the amplified oligonucleotide using any suitable technique to produce an oligonucleotide that contains only the assembly sequence.

In some embodiments, a plurality of different oligonucleotides (e.g., about 5, 10, 50, 100, or more) with different central assembly sequences may have identical 5′ amplification sequences and identical 3′ amplification sequences. These oligonucleotides can all be amplified in the same reaction using the same amplification primers.

A preparation of an oligonucleotide designed to have a certain sequence may include oligonucleotide molecules having the designed sequence in addition to oligonucleotide molecules that contain errors (e.g., that differ from the designed sequence at least at one position). A sequence error may include one or more nucleotide deletions, additions, substitutions (e.g., transversion or transition), inversions, duplications, or any combination of two or more thereof. Oligonucleotide errors may be generated during oligonucleotide synthesis. Different synthetic techniques may be prone to different error profiles and frequencies. In some embodiments, error rates may vary from 1/10 to 1/200 errors per base depending on the synthesis protocol that is used. However, in some embodiments lower error rates may be achieved. Also, the types of errors may depend on the synthetic techniques that are used. For example, in some embodiments chip-based oligonucleotide synthesis may result in relatively more deletions than column-based synthetic techniques.

In some embodiments, one or more oligonucleotide preparations may be processed to remove (or reduce the frequency of) error-containing oligonucleotides. In some embodiments, a hybridization technique may be used wherein an oligonucleotide preparation is hybridized under stringent conditions one or more times to an immobilized oligonucleotide preparation designed to have a complementary sequence. Oligonucleotides that do not bind may be removed in order to selectively or specifically remove oligonucleotides that contain errors that would destabilize hybridization under the conditions used. It should be appreciated that this processing may not remove all error-containing oligonucleotides since many have only one or two sequence errors and may still bind to the immobilized oligonucleotides with sufficient affinity for a fraction of them to remain bound through this selection processing procedure.

In some embodiments, a nucleic acid binding protein or recombinase (e.g., RecA) may be included in one or more of the oligonucleotide processing steps to improve the selection of error free oligonucleotides. For example, by preferentially promoting the hybridization of oligonucleotides that are completely complementary with the immobilized oligonucleotides, the amount of error containing oligonucleotides that are bound may be reduced. As a result, this oligonucleotide processing procedure may remove more error-containing oligonucleotides and generate an oligonucleotide preparation that has a lower error frequency (e.g., with an error rate of less than 1/50, less than 1/100, less than 1/200, less than 1/300, less than 1/400, less than 1/500, less than 1/1,000, or less than 1/2,000 errors per base.

A plurality of oligonucleotides used in an assembly reaction may contain preparations of synthetic oligonucleotides, single-stranded oligonucleotides, double-stranded oligonucleotides, amplification products, oligonucleotides that are processed to remove (or reduce the frequency of) error-containing variants, etc., or any combination of two or more thereof.

In some aspects, synthetic oligonucleotides synthesized on an array (e.g., a chip) are not amplified prior to assembly. In some embodiments, a polymerase-based or ligase-based assembly using non-amplified oligonucleotides may be performed in a microfluidic device.

In some aspects, a synthetic oligonucleotide may be amplified prior to use. Either strand of a double-stranded amplification product may be used as an assembly oligonucleotide and added to an assembly reaction as described herein. A synthetic oligonucleotide may be amplified using a pair of amplification primers (e.g., a first primer that hybridizes to the 3′ region of the oligonucleotide and a second primer that hybridizes to the 3′ region of the complement of the oligonucleotide). The oligonucleotide may be synthesized on a support such as a chip (e.g., using an ink-jet-based synthesis technology). In some embodiments, the oligonucleotide may be amplified while it is still attached to the support. In some embodiments, the oligonucleotide may be removed or cleaved from the support prior to amplification. The two strands of a double-stranded amplification product may be separated and isolated using any suitable technique. In some embodiments, the two strands may be differentially labeled (e.g., using one or more different molecular weight, affinity, fluorescent, electrostatic, magnetic, and/or other suitable tags). The different labels may be used to purify and/or isolate one or both strands. In some embodiments, biotin may be used as a purification tag. In some embodiments, the strand that is to be used for assembly may be directly purified (e.g., using an affinity or other suitable tag). In some embodiments, the complementary strand is removed (e.g., using an affinity or other suitable tag) and the remaining strand is used for assembly.

In some embodiments, a synthetic oligonucleotide may include a central assembly sequence flanked by 5′ and 3′ amplification sequences. The central assembly sequence is designed for incorporation into an assembled nucleic acid. The flanking sequences are designed for amplification and are not intended to be incorporated into the assembled nucleic acid. The flanking amplification sequences may be used as universal primer sequences to amplify a plurality of different assembly oligonucleotides that share the same amplification sequences but have different central assembly sequences. In some embodiments, the flanking sequences are removed after amplification to produce an oligonucleotide that contains only the assembly sequence.

In some embodiments, one of the two amplification primers may be biotinylated. The nucleic acid strand that incorporates this biotinylated primer during amplification can be affinity purified using streptavidin (e.g., bound to a bead, column, or other surface). In some embodiments, the amplification primers also may be designed to include certain sequence features that can be used to remove the primer regions after amplification in order to produce a single-stranded assembly oligonucleotide that includes the assembly sequence without the flanking amplification sequences.

In some embodiments, the non-biotinylated strand may be used for assembly. The assembly oligonucleotide may be purified by removing the biotinylated complementary strand. In some embodiments, the amplification sequences may be removed if the non-biotinylated primer includes a dU at its 3′ end, and if the amplification sequence recognized by (i.e., complementary to) the biotinylated primer includes at most three of the four nucleotides and the fourth nucleotide is present in the assembly sequence at (or adjacent to) the junction between the amplification sequence and the assembly sequence. After amplification, the double-stranded product is incubated with T4 DNA polymerase (or other polymerase having a suitable editing activity) in the presence of the fourth nucleotide (without any of the nucleotides that are present in the amplification sequence recognized by the biotinylated primer) under appropriate reaction conditions. Under these conditions, the 3′ nucleotides are progressively removed through to the nucleotide that is not present in the amplification sequence (referred to as the fourth nucleotide above). As a result, the amplification sequence that is recognized by the biotinylated primer is removed. The biotinylated strand is then removed. The remaining non-biotinylated strand is then treated with uracil-DNA glycosylase (UDG) to remove the non-biotinylated primer sequence. This technique generates a single-stranded assembly oligonucleotide without the flanking amplification sequences. It should be appreciated that this technique may be used to process a single amplified oligonucleotide preparation or a plurality of different amplified oligonucleotides in a single reaction if they share the same amplification sequence features described above.

In some embodiments, the biotinylated strand may be used for assembly. The assembly oligonucleotide may be obtained directly by isolating the biotinylated strand. In some embodiments, the amplification sequences may be removed if the biotinylated primer includes a dU at its 3′ end, and if the amplification sequence recognized by (i.e., complementary to) the non-biotinylated primer includes at most three of the four nucleotides and the fourth nucleotide is present in the assembly sequence at (or adjacent to) the junction between the amplification sequence and the assembly sequence. After amplification, the double-stranded product is incubated with T4 DNA polymerase (or other polymerase having a suitable editing activity) in the presence of the fourth nucleotide (without any of the nucleotides that are present in the amplification sequence recognized by the non-biotinylated primer) under appropriate reaction conditions. Under these conditions, the 3′ nucleotides are progressively removed through to the nucleotide that is not present in the amplification sequence (referred to as the fourth nucleotide above). As a result, the amplification sequence that is recognized by the non-biotinylated primer is removed. The biotinylated strand is then isolated (and the non-biotinylated strand is removed). The isolated biotinylated strand is then treated with UDG to remove the biotinylated primer sequence. This technique generates a single-stranded assembly oligonucleotide without the flanking amplification sequences. It should be appreciated that this technique may be used to process a single amplified oligonucleotide preparation or a plurality of different amplified oligonucleotides in a single reaction if they share the same amplification sequence features described above.

It should be appreciated that the biotinylated primer may be designed to anneal to either the synthetic oligonucleotide or to its complement for the amplification and purification reactions described above. Similarly, the non-biotinylated primer may be designed to anneal to either strand provided it anneals to the strand that is complementary to the strand recognized by the biotinylated primer.

In certain embodiments, it may be helpful to include one or more modified oligonucleotides in an assembly reaction. An oligonucleotide may be modified by incorporating a modified-base (e.g., a nucleotide analog) during synthesis, by modifying the oligonucleotide after synthesis, or any combination thereof. Examples of modifications include, but are not limited to, one or more of the following: universal bases such as nitroindoles, dP and dK, inosine, uracil; halogenated bases such as BrdU; fluorescent labeled bases; non-radioactive labels such as biotin (as a derivative of dT) and digoxigenin (DIG); 2,4-Dinitrophenyl (DNP); radioactive nucleotides; post-coupling modification such as dR-NH₂ (deoxyribose-NH₂); Acridine (6-chloro-2-methoxiacridine); and spacer phosphoramides which are used during synthesis to add a spacer ‘arm’ into the sequence, such as C3, C8 (octanediol), C9, C12, HEG (hexaethlene glycol) and C18.

It should be appreciated that one or more nucleic acid binding proteins or recombinases are preferably not included in a post-assembly fidelity optimization technique (e.g., a screening technique using a MutS or MutS homolog), because the optimization procedure involves removing error-containing nucleic acids via the production and removal of heteroduplexes. Accordingly, any nucleic acid binding proteins or recombinases (e.g., RecA) that were included in the assembly steps are preferably removed (e.g., by inactivation, column purification or other suitable technique) after assembly and prior to fidelity optimization.

### Applications

Aspects of the invention may be useful for a range of applications involving the production and/or use of synthetic nucleic acids. As described herein, the invention provides methods for assembling synthetic nucleic acids with increased efficiency. The resulting assembled nucleic acids may be amplified in vitro (e.g., using PCR, LCR, or any suitable amplification technique), amplified in vivo (e.g., via cloning into a suitable vector), isolated and/or purified. An assembled nucleic acid (alone or cloned into a vector) may be transformed into a host cell (e.g., a prokaryotic, eukaryotic, insect, mammalian, or other host cell). In some embodiments, the host cell may be used to propagate the nucleic acid. In certain embodiments, the nucleic acid may be integrated into the genome of the host cell. In some embodiments, the nucleic acid may replace a corresponding nucleic acid region on the genome of the cell (e.g., via homologous recombination). Accordingly, nucleic acids may be used to produce recombinant organisms. In some embodiments, a target nucleic acid may be an entire genome or large fragments of a genome that are used to replace all or part of the genome of a host organism. Recombinant organisms also may be used for a variety of research, industrial, agricultural, and/or medical applications.

Many of the techniques described herein can be used together, applying combinations of one or more extension-based and/or ligation-based assembly techniques at one or more points to produce long nucleic acid molecules. For example, concerted assembly may be used to assemble oligonucleotide duplexes and nucleic acid fragments of less than 100 to more than 10,000 base pairs in length (e.g., 100 mers to 500 mers, 500 mers to 1,000 mers, 1,000 mers to 5,000 mers, 5,000 mers to 10,000 mers, 25,000 mers, 50,000 mers, 75,000 mers, 100,000 mers, etc.). In an exemplary embodiment, methods described herein may be used during the assembly of an entire genome (or a large fragment thereof, e.g., about 10%, 20%, 30%, 40%, 50%, 60%, 70%, 80%, 90%, or more) of an organism (e.g., of a viral, bacterial, yeast, or other prokaryotic or eukaryotic organism), optionally incorporating specific modifications into the sequence at one or more desired locations.

Any of the nucleic acid products (e.g., including nucleic acids that are amplified, cloned, purified, isolated, etc.) may be packaged in any suitable format (e.g., in a stable buffer, lyophilized, etc.) for storage and/or shipping (e.g., for shipping to a distribution center or to a customer). Similarly, any of the host cells (e.g., cells transformed with a vector or having a modified genome) may be prepared in a suitable buffer for storage and or transport (e.g., for distribution to a customer). In some embodiments, cells may be frozen. However, other stable cell preparations also may be used.

Host cells may be grown and expanded in culture. Host cells may be used for expressing one or more RNAs or polypeptides of interest (e.g., therapeutic, industrial, agricultural, and/or medical proteins). The expressed polypeptides may be natural polypeptides or non-natural polypeptides. The polypeptides may be isolated or purified for subsequent use.

Accordingly, nucleic acid molecules generated using methods of the invention can be incorporated into a vector. The vector may be a cloning vector or an expression vector. A vector may comprise an origin of replication and one or more selectable markers (e.g., antibiotic resistant markers, auxotrophic markers, etc.). In some embodiments, the vector may be a viral vector. A viral vector may comprise nucleic acid sequences capable of infecting target cells. Similarly; in some embodiments, a prokaryotic expression vector operably linked to an appropriate promoter system can be used to transform target cells. In other embodiments, a eukaryotic vector operably linked to an appropriate promoter system can be used to transfect target cells or tissues.

Transcription and/or translation of the constructs described herein may be carried out in vitro (i.e., using cell-free systems) or in vivo (i.e., expressed in cells). In some embodiments, cell lysates may be prepared. In certain embodiments, expressed RNAs or polypeptides may be isolated or purified. Nucleic acids of the invention also may be used to add detection and/or purification tags to expressed polypeptides or fragments thereof. Examples of polypeptide-based fusion/tag include, but are not limited to, hexa-histidine (His⁶) Myc and HA, and other polypeptides with utility, such as GFP, GST, MBP, chitin and the like. In some embodiments, polypeptides may comprise one or more unnatural amino acid residue(s).

In some embodiments, antibodies can be made against polypeptides or fragment(s) thereof encoded by one or more synthetic nucleic acids.

In certain embodiments, synthetic nucleic acids may be provided as libraries for screening in research and development (e.g., to identify potential therapeutic proteins or peptides, to identify potential protein targets for drug development, etc.)

In some embodiments, a synthetic nucleic acid may be used as a therapeutic (e.g., for gene therapy, or for gene regulation). For example, a synthetic nucleic acid may be administered to a patient in an amount sufficient to express a therapeutic amount of a protein. In other embodiments, a synthetic nucleic acid may be administered to a patient in an amount sufficient to regulate (e.g., down-regulate) the expression of a gene.

It should be appreciated that different acts or embodiments described herein may be performed independently and may be performed at different locations in the United States or outside the United States. For example, each of the acts of receiving an order for a target nucleic acid, analyzing a target nucleic acid sequence, identifying an assembly strategy, designing one or more starting nucleic acids (e.g., oligonucleotides), synthesizing starting nucleic acid(s), purifying starting nucleic acid(s), assembling starting nucleic acid(s), isolating assembled nucleic acid(s), confirming the sequence of assembled nucleic acid(s), manipulating assembled nucleic acid(s) (e.g., amplifying, cloning, inserting into a host genome, etc.), and any other acts or any parts of these acts may be performed independently either at one location or at different sites within the United States or outside the United States. In some embodiments, an assembly procedure may involve a combination of acts that are performed at one site (in the United States or outside the United States) and acts that are performed at one or more remote sites (within the United States or outside the United States).

### Automated Applications

Aspects of the invention may include automating one or more acts described herein. For example, a sequence analysis may be automated in order to generate a synthesis strategy automatically. The synthesis strategy may include i) the design of the starting nucleic acids that are to be assembled into the target nucleic acid, ii) the choice of the assembly technique(s) to be used, iii) the number of rounds of assembly and error screening or sequencing steps to include, and/or decisions relating to subsequent processing of an assembled target nucleic acid. Similarly, one or more steps of an assembly reaction may be automated using one or more automated sample handling devices (e.g., one or more automated liquid or fluid handling devices). For example, the synthesis and optional selection of starting nucleic acids (e.g., oligonucleotides) may be automated using a nucleic acid synthesizer and automated procedures. Automated devices and procedures may be used to mix reaction reagents, including one or more of the following: starting nucleic acids, buffers, enzymes (e.g., one or more ligases and/or polymerases), nucleotides, nucleic acid binding proteins or recombinases, salts, and any other suitable agents such as stabilizing agents. In some embodiments, reaction reagents may include one or more reagents or reaction conditions suitable for extension-based assembly, ligation-based assembly, or combinations thereof. Automated devices and procedures also may be used to control the reaction conditions. For example, an automated thermal cycler may be used to control reaction temperatures and any temperature cycles that may be used. In some embodiments, a thermal cycler may be automated to provide one or more reaction temperatures or temperature cycles suitable for incubating nucleic acid fragments prior to transformation. Similarly, subsequent purification and analysis of assembled nucleic acid products may be automated. For example, fidelity optimization steps (e.g., a MutS error screening procedure) may be automated using appropriate sample processing devices and associated protocols. Sequencing also may be automated using a sequencing device and automated sequencing protocols. Additional steps (e.g., amplification, cloning, etc.) also may be automated using one or more appropriate devices and related protocols. It should be appreciated that one or more of the device or device components described herein may be combined in a system (e.g., a robotic system). Assembly reaction mixtures (e.g., liquid reaction samples) may be transferred from one component of the system to another using automated devices and procedures (e.g., robotic manipulation and/or transfer of samples and/or sample containers, including automated pipetting devices, etc.). The system and any components thereof may be controlled by a control system.

Accordingly, acts of the invention may be automated using, for example, a computer system (e.g., a computer controlled system). A computer system on which aspects of the invention can be implemented may include a computer for any type of processing (e.g., sequence analysis and/or automated device control as described herein). However, it should be appreciated that certain processing steps may be provided by one or more of the automated devices that are part of the assembly system. In some embodiments, a computer system may include two or more computers. For example, one computer may be coupled, via a network, to a second computer. One computer may perform sequence analysis. The second computer may control one or more of the automated synthesis and assembly devices in the system. In other aspects, additional computers may be included in the network to control one or more of the analysis or processing acts. Each computer may include a memory and processor. The computers can take any form, as the aspects of the present invention are not limited to being implemented on any particular computer platform. Similarly, the network can take any form, including a private network or a public network (e.g., the Internet). Display devices can be associated with one or more of the devices and computers. Alternatively, or in addition, a display device may be located at a remote site and connected for displaying the output of an analysis in accordance with the invention. Connections between the different components of the system may be via wire, wireless transmission, satellite transmission, any other suitable transmission, or any combination of two or more of the above.

In accordance with one embodiment of the present invention for use on a computer system it is contemplated that sequence information (e.g., a target sequence, a processed analysis of the target sequence, etc.) can be obtained and then sent over a public network, such as the Internet, to a remote location to be processed by computer to produce any of the various types of outputs discussed herein (e.g., in connection with oligonucleotide design). However, it should be appreciated that the aspects of the present invention described herein are not limited in that respect, and that numerous other configurations are possible. For example, all of the analysis and processing described herein can alternatively be implemented on a computer that is attached locally to a device, an assembly system, or one or more components of an assembly system. As a further alternative, as opposed to transmitting sequence information (e.g., a target sequence, a processed analysis of the target sequence, etc.) over a communication medium (e.g., the network), the information can be loaded onto a computer readable medium that can then be physically transported to another computer for processing in the manners described herein. In another embodiment, a combination of two or more transmission/delivery techniques may be used. It also should be appreciated that computer implementable programs for performing a sequence analysis or controlling one or more of the devices, systems, or system components described herein also may be transmitted via a network or loaded onto a computer readable medium as described herein. Accordingly, aspects of the invention may involve performing one or more steps within the United States and additional steps outside the United States. In some embodiments, sequence information (e.g., a customer order) may be received at one location (e.g., in one country) and sent to a remote location for processing (e.g., in the same country or in a different country), for example, for sequence analysis to determine a synthesis strategy and/or design oligonucleotides. In certain embodiments, a portion of the sequence analysis may be performed at one site (e.g., in one country) and another portion at another site (e.g., in the same country or in another country). In some embodiments, different steps in the sequence analysis may be performed at multiple sites (e.g., all in one country or in several different countries). The results of a sequence analysis then may be sent to a further site for synthesis. However, in some embodiments, different synthesis and quality control steps may be performed at more than one site (e.g., within one county or in two or more countries). An assembled nucleic acid then may be shipped to a further site (e.g., either to a central shipping center or directly to a client).

Each of the different aspects, embodiments, or acts of the present invention described herein can be independently automated and implemented in any of numerous ways. For example, each aspect, embodiment, or act can be independently implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as one or more controllers that control the above-discussed functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments of the present invention comprises at least one computer-readable medium (e.g., a computer memory, a floppy disk, a compact disk, a tape, etc.) encoded with a computer program (i.e., a plurality of instructions), which, when executed on a processor, performs one or more of the above-discussed functions of the present invention. The computer-readable medium can be transportable such that the program stored thereon can be loaded onto any computer system resource to implement one or more functions of the present invention discussed herein. In addition, it should be appreciated that the reference to a computer program which, when executed, performs the above-discussed functions, is not limited to an application program running on a host computer. Rather, the term computer program is used herein in a generic sense to reference any type of computer code (e.g., software or microcode) that can be employed to program a processor to implement the above-discussed aspects of the present invention.

It should be appreciated that in accordance with several embodiments of the present invention wherein processes are implemented in a computer readable medium, the computer implemented processes may, during the course of their execution, receive input manually (e.g., from a user).

Accordingly, overall system-level control of the assembly devices or components described herein may be performed by a system controller which may provide control signals to the associated nucleic acid synthesizers, liquid handling devices, thermal cyclers, sequencing devices, associated robotic components, as well as other suitable systems for performing the desired input/output or other control functions. Thus, the system controller along with any device controllers together form a controller that controls the operation of a nucleic acid assembly system. The controller may include a general purpose data processing system, which can be a general purpose computer, or network of general purpose computers, and other associated devices, including communications devices, modems, and/or other circuitry or components necessary to perform the desired input/output or other functions. The controller can also be implemented, at least in part, as a single special purpose integrated circuit (e.g., ASIC) or an array of ASICs, each having a main or central processor section for overall, system-level control, and separate sections dedicated to performing various different specific computations, functions and other processes under the control of the central processor section. The controller can also be implemented using a plurality of separate dedicated programmable integrated or other electronic circuits or devices, e.g., hard wired electronic or logic circuits such as discrete element circuits or programmable logic devices. The controller can also include any other components or devices, such as user input/output devices (monitors, displays, printers, a keyboard, a user pointing device, touch screen, or other user interface, etc.), data storage devices, drive motors, linkages, valve controllers, robotic devices, vacuum and other pumps, pressure sensors, detectors, power supplies, pulse sources, communication devices or other electronic circuitry or components, and so on. The controller also may control operation of other portions of a system, such as automated client order processing, quality control, packaging, shipping, billing, etc., to perform other suitable functions known in the art but not described in detail herein.

### Business Applications

Aspects of the invention may be useful to streamline nucleic acid assembly reactions. Accordingly, aspects of the invention relate to marketing methods, compositions, kits, devices, and systems for increasing nucleic acid assembly throughput involving combinations of one or more extension-based and/or ligation-based assembly techniques described herein.

Aspects of the invention may be useful for reducing the time and/or cost of production, commercialization, and/or development of synthetic nucleic acids, and/or related compositions. Accordingly, aspects of the invention relate to business methods that involve collaboratively (e.g., with a partner) or independently marketing one or more methods, kits, compositions, devices, or systems for analyzing and/or assembling synthetic nucleic acids as described herein. For example, certain embodiments of the invention may involve marketing a procedure and/or associated devices or systems involving nucleic acid assembly techniques described herein. In some embodiments, synthetic nucleic acids, libraries of synthetic nucleic acids, host cells containing synthetic nucleic acids, expressed polypeptides or proteins, etc., also may be marketed.

Marketing may involve providing information and/or samples relating to methods, kits, compositions, devices, and/or systems described herein. Potential customers or partners may be, for example, companies in the pharmaceutical, biotechnology and agricultural industries, as well as academic centers and government research organizations or institutes. Business applications also may involve generating revenue through sales and/or licenses of methods, kits, compositions, devices, and/or systems of the invention.

### EXAMPLES

### Example 1

### Nucleic Acid Fragment Assembly

Gene assembly via a 2-step PCR method: In step (1), a primerless assembly of oligonucleotides is performed and in step (2) an assembled nucleic acid fragment is amplified in a primer-based amplification.

A 993 base long promoter>EGFP construct was assembled from 50-mer abutting oligonucleotides using a 2-step PCR assembly.

Mixed oligonucleotide pools were prepared as follows: 36 overlapping 50-mer oligonucleotides and two 5′ terminal 59-mers were separated into 4 pools, each corresponding to overlapping 200-300 nucleotide segments of the final construct. The total oligonucleotide concentration in each pool was 5 μM.

A primerless PCR extension reaction was used to stitch (assemble) overlapping oligonucleotides in each pool. The PCR extension reaction mixture was as follows:

| | |
|---|---|
| oligonucleotide pool (5 μM total) | 1.0 μl (~25 nM final each) |
| dNTP (10 mM each) | 0.5 μl (250 μM final each) |
| Pfu buffer (10x) | 2.0 μl |
| Pfu polymerase (2.5 U/μl) | 0.5 μl |
| dH₂O to 20 μl | |

Assembly was achieved by cycling this mixture through several rounds of denaturing, annealing, and extension reactions as follows:
start 2 min. 95° C.
30 cycles of 95° C. 30 sec., 65° C. 30 sec., 72° C. 1 min.
final 72° C. 2 min. extension step

The resulting product was exposed to amplification conditions to amplify the desired nucleic acid fragments (sub-segments of 200-300 nucleotides). The following PCR mix was used:

| | |
|---|---|
| primerless PCR product | 1.0 μl |
| primer 5′ (1.2 μM) | 5 μl (300 nM final) |
| primer 3′ (1.2 μM) | 5 μl (300 nM final) |
| dNTP (10 mM each) | 0.5 μl (250 μM final each) |
| Pfu buffer (10x) | 2.0 μl |
| Pfu polymerase (2.5 U/μl) | 0.5 μl |
| dH₂O to 20 μl | |

The following PCR cycle conditions were used:
start 2 min. 95° C.
35 cycles of 95° C. 30 sec., 65° C. 30 sec., 72° C. 1 min.
final 72° C. 2 min. extension step

The amplified sub-segments were assembled using another round of primerless PCR as follows. A diluted amplification product was prepared for each sub-segment by diluting each amplified sub-segment PCR product 1:10 (4 μl mix+36 μl dH₂O). This diluted mix was used as follows:

| | |
|---|---|
| diluted sub-segment mix | 1.0 μl |
| dNTP (10 mM each) | 0.5 μl (250 μM final each) |
| Pfu buffer (10x) | 2.0 μl |
| Pfu polymerase (2.5 U/μl) | 0.5 μl |
| dH₂O to 20 μl | |

The following PCR cycle conditions were used:
start 2 min. 95° C.
30 cycles of 95° C. 30 sec., 65° C. 30 sec., 72° C. 1 min.
final 72° C. 2 min. extension step

The full-length 993 nucleotide long promoter>EGFP was amplified in the following PCR mix:

| | |
|---|---|
| assembled sub-segments | 1.0 μl |
| primer 5′ (1.2 μM) | 5 μl (300 nM final) |
| primer 3′ (1.2 μM) | 5 μl (300 nM final) |
| dNTP (10 mM each) | 0.5 μl (250 μM final each) |
| Pfu buffer (10x) | 2.0 μl |
| Pfu polymerase (2.5 U/μl) | 0.5 μl |
| dH₂O to 20 μl | |

The following PCR cycle conditions were used:
start 2 min. 95° C.
35 cycles of 95° C. 30 sec., 65° C. 30 sec., 72° C. 1 min.
final 72° C. 2 min. extension step

### EQUIVALENTS

The present invention provides among other things methods for assembling large polynucleotide constructs and organisms having increased genomic stability. While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

### INCORPORATION BY REFERENCE

All publications, patents and sequence database entries mentioned herein, including those items listed below, are hereby incorporated by reference in their entirety as if each individual publication or patent was specifically and individually indicated to be incorporated by reference. In addition, the disclosures of copending provisional application Ser. No. 60/801,833, filed May 19, 2006, and Ser. No. 60/801,834, filed May 19, 2006, and the utility and PCT applications claiming priority thereto. In case of conflict, the present application, including any definitions herein, will control.

1. A method of synthesizing a target nucleic acid, the method comprising:
analyzing a sequence of a target nucleic acid for the presence of one or more predetermined interfering sequence features that are predicted to disrupt a multiplex oligonucleotide assembly reaction,
assembling one or more first fragments of the target nucleic acid using a polymerase-mediated oligonucleotide assembly reaction, wherein no predetermined interfering sequence features are present in any of the one or more first fragments,
if one or more predetermined interfering sequence features are present in the target nucleic acid, assembling one or more second fragments of the target nucleic acid using a ligase-mediated oligonucleotide assembly reaction, wherein the one or more second fragments comprise the one or more predetermined interfering sequence features, and
assembling the target nucleic acid from the one or more first fragments and the one or more second fragments if the target nucleic acid contained one or more predetermined interfering sequence features.

2. A method of designing a target nucleic acid assembly, the method comprising:
analyzing a sequence of a target nucleic acid for the presence of one or more predetermined interfering sequence features that are predicted to disrupt a multiplex oligonucleotide assembly reaction,
selecting one or more first fragments of the target nucleic acid for assembly in a polymerase-mediated oligonucleotide assembly reaction, wherein no predetermined interfering sequence features are present in any of the one or more first fragments, and
if one or more predetermined interfering sequence features are present in the target nucleic acid, selecting one or more second fragments of the target nucleic acid for assembly in a ligase-mediated oligonucleotide assembly reaction, wherein the one or more second fragments comprise the one or more predetermined interfering sequence features,
wherein the target nucleic acid can be assembled from the one or more first fragments and the one or more second fragments if they are selected.

3. The method of claim 2, further comprising the act of assembling the one or more first fragments.

4. The method of claim 3, further comprising the act of assembling the one or more second fragments.

5. The method of claim 2, further comprising the act of assembling the target nucleic acid.

6. The method of claim 1, wherein each of the one or more predetermined interfering sequence features is, independently, a region of high GC content, a region of low GC content, a region containing a repeated sequence, a region containing a partially repeated sequence, a region containing a polymerase stall sequence, a region containing a sequence likely to be replicated erroneously by a polymerase, or any other sequence feature predicted to disrupt a multiplex oligonucleotide assembly reaction.

7. The method of claim 6, wherein the region of high GC content contains greater than 70% GC over a length of at least 10 to 20 nucleotides.

8. The method of claim 6, wherein the region of low GC content contains less than 30% GC over a length of at least 10-20 nucleotides.

9. The method of claim 6, wherein the repeated sequence is at least 7 bases long.

10. The method of claim 6, wherein the repeated sequence is a direct repeat.

11. The method of claim 6, wherein the repeated sequence is an inverted repeat.

12. The method of claim 1, wherein the act of analyzing the sequence of the target nucleic acid comprises calculating hybridization binding energies between pairs of test sequences selected from different regions of the target nucleic acid.

13. The method of claim 12, wherein each test sequence is a randomly selected sequence of between 20 and 60 consecutive bases of the target nucleic acid.

14. The method of claim 12, wherein each test sequence is selected according to a computer-implemented algorithm.

15. The method of claim 12, wherein an interfering sequence feature is the presence of a pair of test sequences having a predicted hybridization binding energy greater than a threshold level.

16. The method of claim 1, wherein the act of analyzing the sequence of the target nucleic acid comprises searching for potential stem-loop structures between pairs of test sequences selected from different regions of the target nucleic acid.

17. The method of claim 16, wherein each test sequence is a randomly selected sequence of between 20 and 60 consecutive bases of the target nucleic acid.

18. The method of claim 16, wherein each test sequence is selected according to a computer-implemented algorithm.

19. The method of claim 14, wherein an interfering sequence feature is the presence of a pair of test sequences that are predicted to form a stem-loop structure wherein at most two bases are unpaired at the 3′ end of one of the sequences.

20-55. (canceled)

56. A system for assembling a target nucleic acid, the system comprising:
a means for analyzing a sequence of a target nucleic acid for the presence of one or more predetermined interfering sequence features that are predicted to disrupt a multiplex oligonucleotide assembly reaction,
a means for assembling one or more first fragments of the target nucleic acid using a polymerase-mediated oligonucleotide assembly reaction, wherein no predetermined interfering sequence features are present in any of the one or more first fragments,
a means for assembling one or more second fragments of the target nucleic acid using a ligase-mediated oligonucleotide assembly reaction if one or more predetermined interfering sequence features are present in the target nucleic acid, wherein the one or more second fragments comprise the one or more predetermined interfering sequence features, and
a means for assembling the target nucleic acid from the one or more first and second fragments.

57-61. (canceled)
